(19) United States
(12) Patent Application Publication
(10) Pub. No.: US 2005/0119301 A1
(43) Pub. Date: Jun. 2, 2005

(54) Treatment of restenosis

(76) Inventors: Alan Husband, New South Wales (AU); Graham Edmund Kelly, New South Wales (AU)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/471,668

(22) PCT Filed: Mar. 15, 2002
(86) PCT No.: PCT/AU02/00288

(30) Foreign Application Priority Data
Mar. 16, 2001 (AU) PR 3770
Jun. 26, 2001 (AU) PR 9926

(51) Int. Cl. A61K 31/47; A61K 31/353; A61K 31/403

(52) U.S. Cl. 514/312; 514/456; 514/685; 514/284; 514/411

(57) ABSTRACT

This invention relates to a method for inhibiting expression or activity of an adhesion molecule associated with an endothelial cell by contacting the adhesion molecule or endothelial cell with one or more isoflavone compounds or derivatives thereof. The invention also relates to a method of preventing or reducing the risk of restenosis after angioplasty, and to a method for the treatment or prophylaxis of atherosclerosis, coronary artery diseases, other cardiovascular diseases and inflammatory diseases mediated by adhesion molecules. The invention further relates to pharmaceutical compositions useful in these methods, and to methods for the manufacture of such medicaments.

### FIELD OF THE INVENTION

This invention generally relates to a method for inhibiting expression or activity of an adhesion molecule associated with an endothelial cell by contacting the adhesion molecule or endothelial cell with one or more isoflavone compounds or derivatives thereof. The invention also generally relates to a method of preventing or reducing the risk of restenosis after angioplasty, and to a method for the treatment or prophylaxis of atherosclerosis, coronary artery diseases, other cardiovascular diseases and inflammatory diseases mediated by adhesion molecules. The invention further generally relates to pharmaceutical compositions useful in these methods, and to methods for the manufacture of such medicaments. Further aspects of the invention will become apparent from the description, which follows.

### BACKGROUND OF THE INVENTION

Note: Bibliographic details of the publications referred to by author in this specification are colleted at the end of the description.

Atherosclerosis is a chronic inflammatory disease of the arterial intima characterised by the focal accumulation of leukocytes, smooth muscle cells, lipids and extra cellular matrix. Deposits of lipids and other blood derivatives in blood vessel walls, especially of the large arteries, results in the formation of plaques. As the adherence of lipids and leukocytes to the blood vessel wall continues, there is a concomitant thickening of the vessel wall. The size increase of the plaque gains a stenosing character which becomes responsible for vascular occlusions by atheroma, thrombosis or embolism. Serious vascular problems can result including infarction, cardiac insufficiency, stroke and sudden death.

An important predisposing factor to the development of atherosclerosis is the level of cholesterol in the blood. Atheromatous plaque is composed primarily of cells containing cholesterol; consequently a high blood cholesterol level is associated with increased risk of atherosclerosis. While a high absolute cholesterol level is an important risk factor, the risk is more specifically associated with the type of lipoprotein present in the blood. The widespread medical view is that in terms of risk of development of atherosclerosis, low density (LDL) and very low density (VLDL) lipoproteins are detrimental, whereas high density lipoprotein (HDL) has been shown to be a beneficial factor. It follows that the ratio of HDL to LDL in the blood stream is the important factor. The higher the ratio of HDL to LDL, the more a patient appears to be protected against developing atherosclerosis, even if their total cholesterol level is slightly elevated. Clinical studies conducted in many centres and countries support this view.

Many clinical and epidemiological studies have identified a high blood level of total cholesterol, and more particularly a high blood level of low density cholesterol, and more particularly a high LDL:HDL cholesterol ratio, as prime risk factors in the development of atherosclerosis. This has led to a variety of therapeutic strategies designed to reduce this risk. There are two broad strategies which show varying degrees of success. The first is the use of drugs that interfere with cholesterol synthesis. The second strategy is to reduce cholesterol absorption from the gut by the use of resins, thereby reducing the pool of cholesterol available within the body. Generally though, neither of these two strategies is optimal because of resulting adverse side effects and limited efficacy.

The development of atherosclerosis is progressed by the oxidation of LDL cholesterol in the arterial wall, leading to an inflammatory lesion. If left unchecked the inflammatory process proceeds until the lesion causes vascular construction, obstruction and infarct.

Antioxidants are molecules or compounds that can inhibit oxidation (i.e. damage) by chemically inactivating the free radicals produced during normal physiological function. If the body is lacking in a supply of antioxidants, some free radicals remain active and attack healthy cells or convert safe compounds into damaging ones. Antioxidants play many important roles in cardiovascular disease. They have attracted particular interest as potential inhibitors of atherosclerosis via inhibition of the oxidative modification of the low density lipoprotein (LDL). Lipoproteins are the major carriers of cholesterol in the body. The majority of the cholesterol associates with LDL to be distributed throughout the body from the liver. The high density lipoprotein (HDL) “mops up” excess cholesterol present in the blood and returns it to the liver. Lipoproteins are very susceptible to oxidation, and once oxidised can accumulate in healthy arterial and vascular walls without being metabolised. Such gathering of lipoproteins greatly increases the risk of atherosclerosis. In particular, the oxidation of LDL in the sub-endothelial space represent an early and causative step in atherogenesis (Steinberg et al., 1989). Thus inhibition of ‘oxidized LDL’ formation by antioxidants is generally thought to slow down the progression of the disease. This is another area of protection in cardiovascular disease that cardioprotective agents can have utility, and in which more and better agents are sought.

Another area in the development of vascular disease and the formation of plaques is the role played by cell adhesion molecules, as reviewed by Hillis and Flapan (1998). Cell adhesion molecules are involved in the adhesion of leukocytes and monocytes to tissues including the vascular endothelium. Known cell adhesion molecules include intercellular adhesion molecules (ICAM 1, 2 and 3), vascular cell-adhesion molecule-1 (VCAM-1) and platelet endothelial cell adhesion molecule-1 (PECAM-1). The role of adhesion molecules in the pathogenesis of cardiovascular disease is supported by the observation that cell adhesion molecules are expressed in atherosclerotic lesions. In addition, cell adhesion molecules are upregulated by several coronary heart disease risk factors and antibodies to cell adhesion molecules are believed to prevent reperfusion injury in animal models. Accordingly, modulation of the expression or activity of adhesion molecules associated with endothelial cells may be useful in the treatment or prevention of cardiovascular pathology by limiting the development of atherosclerotic plaque.

Other molecules such as E-selectin, P-selectin and L-selectin exhibit an adhesion function. In particular, E-selectin is a cell surface protein inducibly expressed in cytokine-activated endothelial cells in response to inflammatory factors such as occurs with tissue injury. The expression of E-selectin by endothelial cells can also be induced by inflammatory factors including interleukin-1 (IL-1), tumour necrosis factor-α (TNF-α) and various endotoxins. Expression of E-selectin is associated with the binding of endothelial cells and platelets with leukocytes and lipids. The binding of leukocytes to endothelial cells is observed at an early stage after tissue injury and is associated with various acute and chronic inflammation. Suppression or inhibition of the expression or activity of adhesion molecules associated with endothelial cells will limit the focal accumulation and adhesion of leukocytes to vessel walls, particularly at areas of injury, damage or infection.

In the treatment of atherosclerosis, angioplasty has been developed to permit non-surgical intervention of the atherosclerotic plaque. During angioplasty treatment dilation of the blood vessel beyond its ability to recoil completely with a balloon catheter increases the vessel lumen, thereby allowing for increased blood flow. This procedure however causes mechanical injury of the arterial wall, following which restenosis commonly occurs. In fact, restenosis is a major problem following traumatic injury rendered to vessels during vascular surgery and treatment.

To counter the onset of restenosis, various other treatments have been proposed including surgical intervention and drug and gene therapy. Such therapies include the administration of compounds which arrest cell division and hyperproliferative disorders, necrose vascular smooth-muscle cells and block the expression of endothelial cell adhesion molecules. Further compounds useful in addressing restenosis include lipid lowering agents, anti-platelet agents, anti-thrombotic agents, calcium channel blockers, angiotensin converting enzyme (ACE) inhibitors and β-blockers. Generally though, as therapeutic agents are not selective, there are often side effects which need to be monitored or countered. In this regard reference is made to drugs currently in use such as Ticlid (ticlopidine), Plavix (clopidrogel) and Cardiprin (aspirin). Typically gastrointestinal disturbances and skin rashes are commonly reported side effects with their use. Other agents such as heparin reportedly inhibits smooth muscle cell proliferation in vitro but when used in vivo has the adverse side effect of inhibiting coagulation.

Given that vascular disease is currently a leading cause of death in today's society, there is a strong need to identify new, improved, better and alternative methods and pharmaceutical agents for its treatment and prevention.

Thus it is a preferred object of the present invention to provide a method for the treatment, amelioration or prophylaxis of vascular and inflammatory diseases, and in particular restenosis associated with vascular intervention. It is a further preferred object of the present invention to provide pharmaceutical compositions for the treatment, amelioration or prophylaxis of vascular and inflammatory diseases, in particular cardiovascular diseases. It is still another preferred object of the present invention to provide methods and compositions to inhibit the expression or activity of adhesion molecules in endothelial cells.

### SUMMARY OF THE INVENTION

Surprisingly the present inventors have found that isoflavone compounds, metabolites and derivatives thereof are particularly useful for inhibiting or down-regulating the expression or activity of adhesion molecules in endothelial cells. It has also been found that isoflavones and derivatives thereof are particularly useful for inhibiting endothelial cell surface adhesion molecules, and in particular E-selectin and VCAM-1.

The present inventors have also surprisingly found that isoflavones and derivatives thereof find use in preventing or reducing the risks of restenosis associated with vascular intervention including angioplasty treatment of atherosclerosis, and the resultant mechanical injury at the angioplasty site during treatment of an atherosclerotic lesion.

Isoflavone compounds, metabolites, derivatives and analogues thereof useful in the methods of the present invention are depicted by the general formula I as set out below.

Thus, according to a first aspect of the invention there is provided a method for inhibiting expression or activity of an adhesion molecule associated with an endothelial cell, which method comprises the step of contacting the adhesion molecule or endothelial cell with one or more compounds of formula I in an amount sufficient to inhibit said expression or activity.

Preferably the adhesion molecule is E-selectin or vascular cell surface adhesion molecule (VCAM-1).

According to a second aspect of the invention there is provided a method for inhibiting the expression or activity of adhesion molecules associated with endothelial cells in a subject, which method comprises the step of administering to the subject a therapeutically effective amount of one or more compounds of formula I.

According to a third aspect of the invention there is provided a method of treating a disease mediated by expression or activity of adhesion molecules associated with endothelial cells in a subject, which method comprises the step of administering to the subject one or more compounds of formula I in an amount sufficient to inhibit said expression or activity of the adhesion molecules associated with the endothelial cells.

Preferably the disease is a vascular disease including restenosis, inflammatory disease, coronary artery disease, angina or small vessel disease, more preferably post-angioplasty restenosis.

According to a fourth aspect of the invention there is provided a method for the treatment, amelioration, prophylaxis or reduction in the risk of restenosis in a subject, which method comprises the step of administering to the subject a therapeutically effective amount of one or more compounds of formula I.

Typically the restenosis is associated with vascular intervention such as coronary intervention. Preferably the vascular coronary intervention is percutaneous transluminal coronary angioplasty, direction coronary atherectomy or stent, more preferably angioplasty.

According to a fifth aspect of the invention there is provided a method for the treatment of procedural vascular trauma in a subject, which method comprises the step of administering to the subject a therapeutically effective amount of one or more compounds of formula I.

Preferably the procedural vascular trauma is angioplasty, vascular surgery, graft or transplant procedure.

According to a sixth aspect of the invention there is provided a method for the treatment or prophylaxis of vascular disease in a subject, which method comprises the step of administering to the subject a therapeutically effective amount of one or more compounds of formula I.

Preferably the vascular disease is restenosis, inflammatory disease, coronary artery disease, angina or small vessel disease, more preferably post-angioplasty restenosis.

According to a seventh aspect of the invention there is provided a pharmaceutical composition in a dosage form suitable for use in the treatment of a disease mediated by expression or activity of adhesion molecules associated with endothelial cells in a subject, which composition comprises one or more compounds of formula I in association with a pharmaceutical acceptable carrier.

According to an eighth aspect of the invention there is provided a pharmaceutical composition in a dosage form suitable for use in preventing or reducing the risk of vascular disease in a subject, which composition comprises one or more compounds of formula I in association with a pharmaceutical acceptable carrier.

According to a ninth aspect of the invention there is provided the use of one or more compounds of Formula I in the manufacture of a medicament for the treatment of a disease mediated by expression or activity of adhesion molecules associated with endothelial cells.

According to a tenth aspect of the invention there is provided the use of one or more compounds of formula I in the manufacture of a medicament for the treatment of vascular disease and/or procedural vascular trauma.

These and other aspects of the invention will become evident from the description and claims which follow, together with the accompanying drawings.

Throughout this specification and the claims which follow, unless the text requires otherwise, the word “comprise”, and variations such as “comprises” or “comprising”, will be understood to imply the inclusion of a stated integer or step or group of integers or steps but not the exclusion of any other integer or step or group of integers or steps.

### BRIEF DESCRIPTION OF THE DRAWINGS

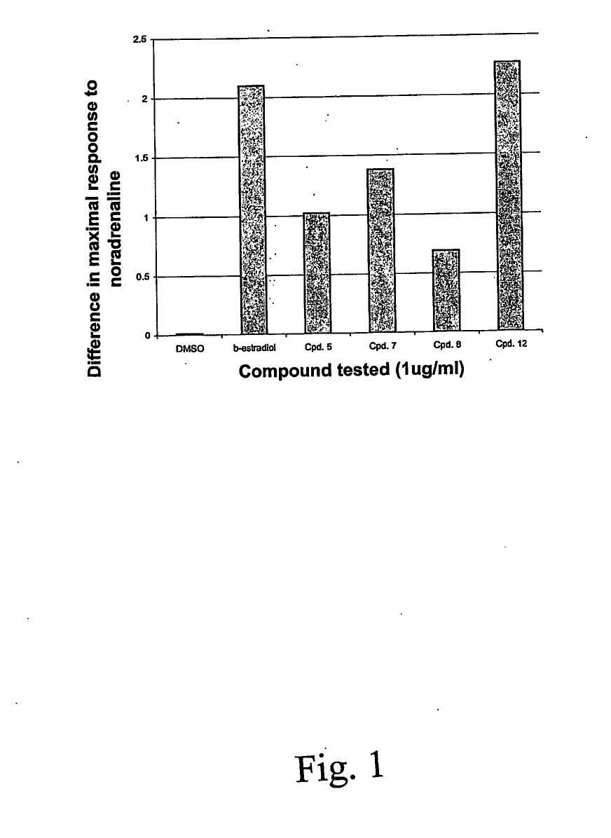

Accompanying FIGS. 1-21 illustrate aspects of the present invention, but are not to be taken as unnecessarily limiting. A description of each of the Figures is provided throughout the Examples which follow.

### DETAILED DESCRIPTION OF THE INVENTION

The term “stenosis” is taken in its broadest sense to mean a narrowing or constriction of the diameter of a bodily passage or orifice, such as in particular a blood vessel or artery, and generally leads to reduced blood flow and the concomitant problems of vessel occlusion. Typically stenosis occurs as a result of the focal accumulation and deposit of lipids and other blood derivatives.

The term “restenosis” or re-stenosis or secondary stenosis is taken in its broadest sense to mean a recurrence of stenosis typically after vascular intervention, injury or surgery, including balloon catheter treatment. Stenosis and restenosis can occur in blood vessels throughout the body, and of particular medical importance is the life threatening and often fatal effects of stenosis in the coronary arteries.

It has been surprisingly discovered that isoflavones and derivatives thereof block the induced expression of the endothelial cell surface adhesion molecules, in particular E-selection and VCAM-1, in response to many signals known to be active in atherosclerosis, restenosis, inflammatory response and other diseases mediated by cell adhesion: molecule expression. This result indicates that isoflavone compounds and derivatives thereof are useful in the treatment or prophylaxis of restenosis, coronary artery diseases, angina and other vascular and cardiovascular diseases, inflammatory diseases mediated by adhesion molecules E-selectin and VCAM-1. The specific molecular mechanisms by which the isoflavones and derivatives function in inhibiting cell adhesion molecule expression are not fully understood.

It has further been shown that isoflavones and derivatives thereof inhibit cellular proliferation of human vascular smooth muscle cells, and also inhibit PDGF-induced Erk activation in human vascular smooth muscle cells. This activity shows the potential for isoflavones and derivatives thereof to prevent the development and progression of atherosclerotic lesions, providing a potential benefit in vascular protection

Isoflavones and derivatives thereof have also been shown to inhibit endothelial cell proliferation and to inhibit endothelial cell migration, and thus have potential for use as cardioprotective therapeutics including the treatment, amelioration or prophylaxis of restenosis after vascular intervention.

Isoflavones and derivatives thereof have also been shown to have potent vascular regulatory capacity. Thus isoflavones and derivatives thereof are capable of antagonising contractile activity, antagonising direct vasodilatation, and protecting against endothelium damage by oxidised low density lipoprotein. While comparable to the ovarian steroid 17β-estradiol, their activities also appear to be unique in their mechanism of action. Thus the isoflavones and derivatives again show potential for use as cardioprotective therapeutics including the treatment, amelioration or prophylaxis of restenosis after vascular intervention

The isoflavone compounds and derivatives can also be administered to treat small vessel disease that is not treatable by surgery or angioplasty, or other vessel disease in which surgery is not an option, and to stabilise patients prior to and after revascularisation therapy. The active compounds can also be administered in the period immediately prior to and following vascular intervention such as coronary or vascular angioplasty as a means to reduce or eliminate the abnormal proliferative and inflammatory response that currently leads to clinically significant restenosis. Further, isoflavones can also be used in the treatment of cardiac transplant rejection and vascular graft and transplant procedures.

The methods of this invention represent a significant advance in treating vascular conditions and disease, in that they go beyond well known therapies designed simply to inhibit the progression of disease. The experimental data provided herein has unexpectedly shown that restenosis after vascular intervention or angioplasty is inhibited or at least markedly reduced in various mammalian animal models. Thus, when used appropriately, the present methods demonstrate the potential of isoflavone compounds and derivatives thereof to medically address restenosis, and possibly to cure atherosclerosis by preventing new lesions from developing and causing established lesions to stabilise or regress.

The isoflavones encompass a class of phytoestrogens found predominantly in leguminous plants with estrogenic activity related to their structural similarity with steroidal estrogen. It is only until recently that the importance of isoflavones has been recognised by its apparent association with a reduction in risk of developing osteoporosis, some forms of cancer and its ability to lower cholesterol levels and blood pressure in mammals. It has also been suggested that many of the biological effects of isoflavones can been accounted for by their conversion in vivo to various other active metabolites. Given the importance of isoflavone metabolites, synthetic derivatives of isoflavones and metabolites are also seen as important therapeutic agents. Further isoflavones have been ingested for years as components of staple foods being very well tolerated with little or no known side effects, unlike many known symptomatic remedies of vascular diseases and related surgery.

Isoflavone compounds, metabolites, derivatives and analogues thereof useful in the methods of the present invention are depicted by the general formula I: 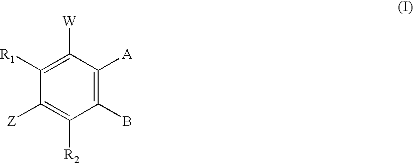
in which
R₁, R₂ and Z are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₂ is as previously defined, and R₁ and Z taken together with the carbon atoms to which they are attached form a five-membered ring selected from 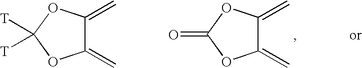
R₁ is as previously defined, and R₂ and Z taken together with the carbon atoms to which they are attached form a five-membered ring selected from 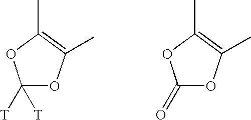
   and
W is R₁, A is hydrogen, hydroxy, NR₃R₄ or thio, and B is selected from 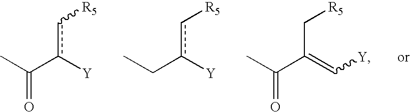
W is R₁, and A and B taken together with the carbon atoms to which they are attached form a six-membered ring selected from 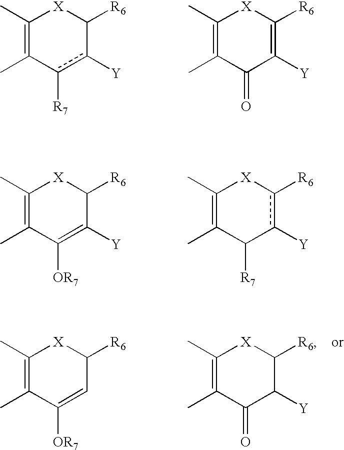
W, A and B taken together with the groups to which they are associated are selected from 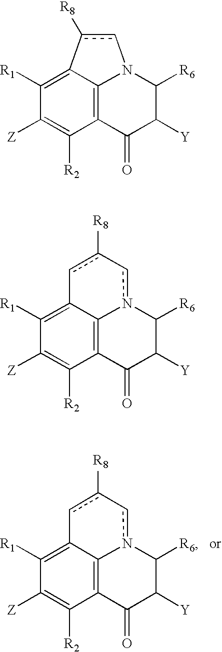
W and A taken together with the groups to which they are associated are selected from 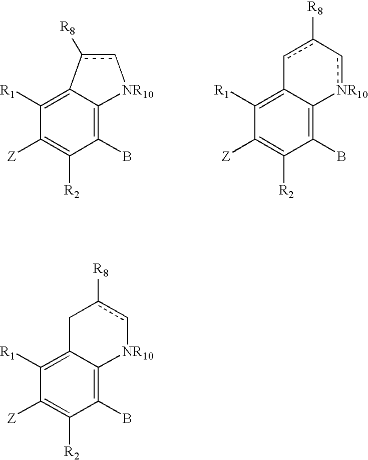
   and B is selected from 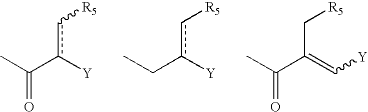
   wherein
R₃ is hydrogen, alkyl, arylalkyl, alkenyl, aryl, an amino acid, C(O)R₁₁, where R₁₁ is hydrogen, alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl, or
R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, alkenyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino,
the drawing 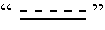
   represents either a single bond or a double bond,
T is independently hydrogen, alkyl or aryl,
X is O, NR₄ or S, and
Y is 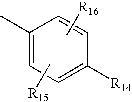
   wherein
R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, thio, alkylthio, amino, alkylamino, dialkylamino, nitro or halo,
including pharmaceutically acceptable salts thereof.

The invention in particular relates to the use of compounds of the general formulae II-VII: 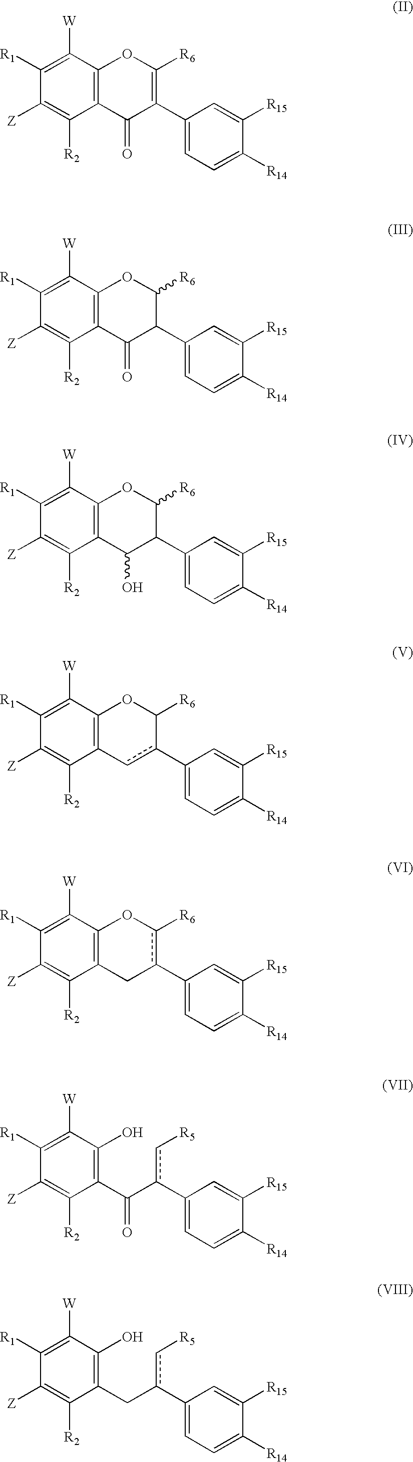
in which
R₁, R₂, R₅, R₆, R₁₄, R₁₅, W and Z are as defined above,
more preferably
R₁, R₂, R₁₄, R₁₅, W and Z are independently hydrogen, hydroxy, OR₉, OC(O)R₁₀, C(O)R₁₀, COOH, CO₂R₁₀, alkyl, haloalkyl, arylalkyl, aryl, thio, alkylthio, amino, alkylamino, dialkylamino, nitro or halo,
R₅ is hydrogen, C(O)R₁₁ where R₁₁ is hydrogen, alkyl, aryl, or an amino acid, or CO₂R₁₂ where R₁₂ is hydrogen, alkyl or aryl,
R₆ is hydrogen, hydroxy, alkyl, aryl, COR₁₁ where R₁₁ is as previously defined, or CO₂R₁₂ where R₁₂ is as previously defined,
R₉ is alkyl, haloalkyl, arylalkyl, or C(O)R₁₁ where R₁₁ is as previously defined, and
R₁₀ is hydrogen, alkyl, amino, aryl, an amino acid, alkylamino or dialkylamino,
more preferably
R₁ and R₁₄ are independently hydroxy, OR₉, OC(O)R₁₀ or halo,
R₂, R₁₅, W and Z are independently hydrogen, hydroxy, OR₉, OC(O)R₁₀, C(O)R₁₀, COOH, CO₂R₁₀, alkyl, haloalkyl, or halo,
R₅ is hydrogen, C(O)R₁₁ where R₁₁ is hydrogen or alkyl, or CO₂R₁₂ where R₁₂ is hydrogen or alkyl,
R₆ is hydrogen or hydroxy,
R₉ is alkyl, arylalkyl or C(O)R₁₁ where R₁₁ is as previously defined, and
R₁₀ is hydrogen or alkyl,
and more preferably
R₁ and R₁₄ are independently hydroxy, methoxy, benzyloxy, acetyloxy or chloro,
R₂, R₁₅, W and Z are independently hydrogen, hydroxy, methoxy, benzyloxy, acetyloxy, methyl, trifluoromethyl or chloro,
R₅ is hydrogen or CO₂R₁₂ where R₁₂ is hydrogen or methyl, and
R₆ is hydrogen.

Particularly preferred compounds of the present invention are selected from:

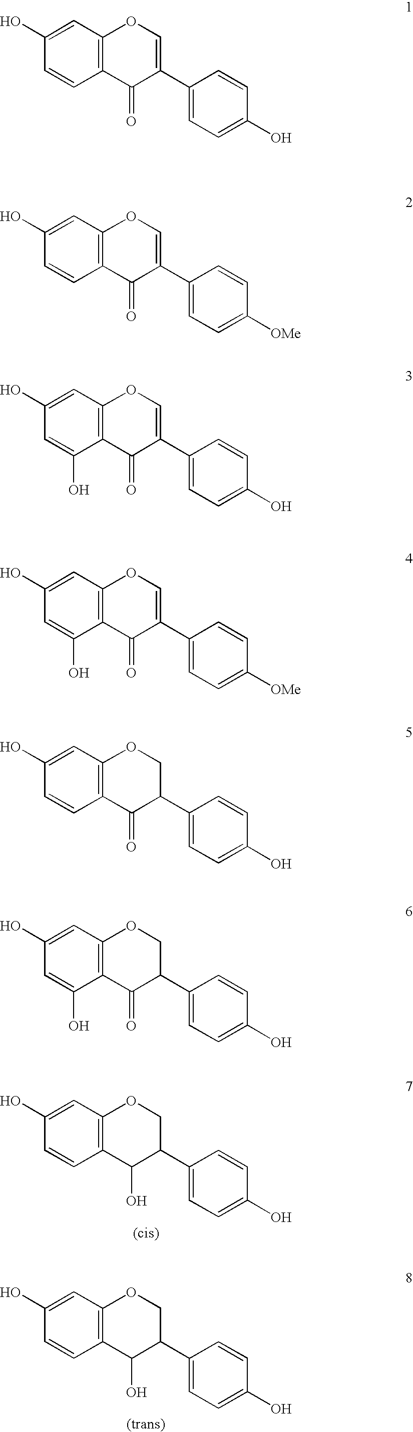

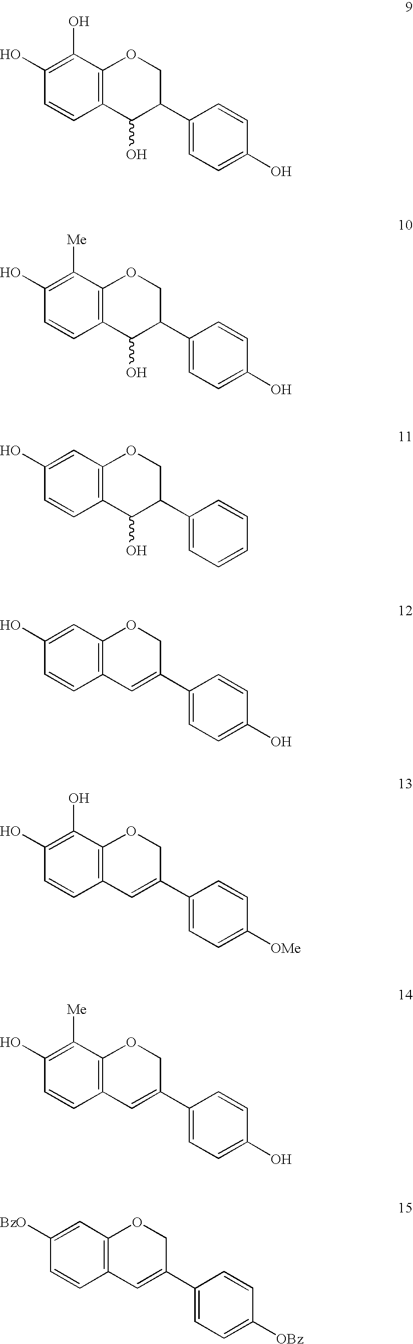

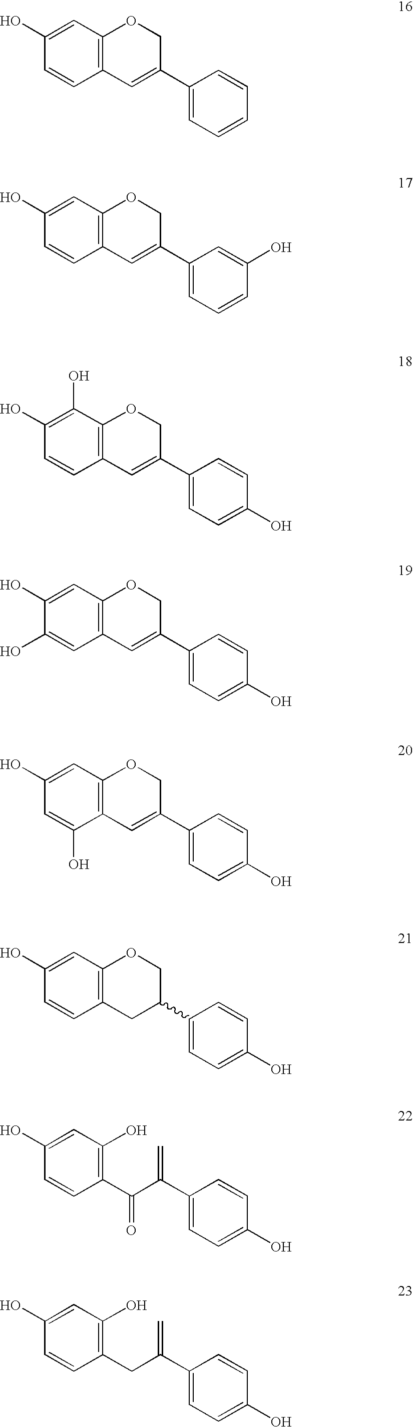

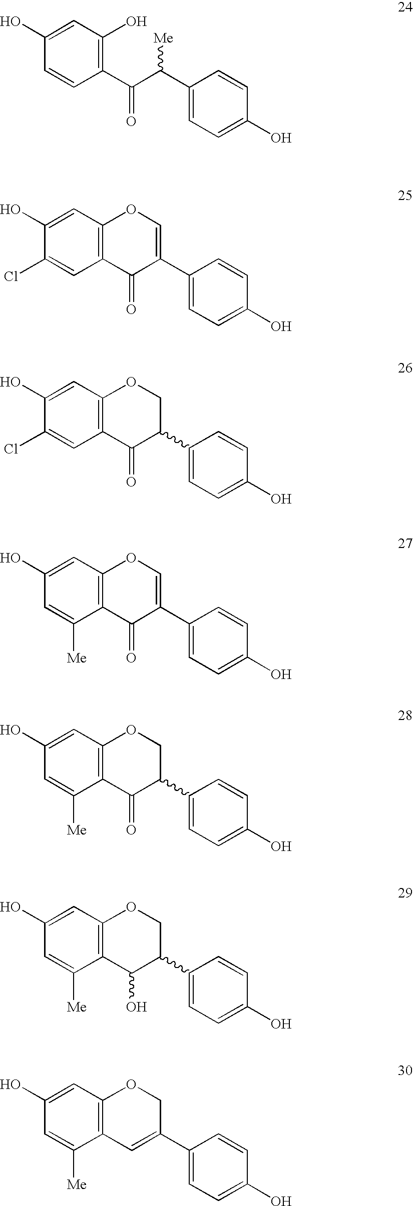

The preferred compounds of the present invention also include all derivatives with physiologically cleavable leaving groups that can be cleaved in vivo from the isoflavone or derivative molecule to which it is attached. The leaving groups include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted isoflavones and derivatives thereof are readily cleavable to the corresponding hydroxy substituted compounds. In addition, the protection of functional groups on the isoflavone compounds and derivatives of the present invention can be carried out by well established methods in the art, for example as described in T. W. Greene (1981).

Most preferred isoflavone compounds contemplated for use in accordance with the invention include formononetin, biochanin, genistein, daidzein and equol, and functional derivatives, equivalents or analogues thereof. Similarly important compounds are the isoflavone metabolites including dihydrodaidzein, cis- and trans-tetrahydrodaidzein and dehydroequol, and derivatives and prodrugs thereof.

Chemical and functional equivalents of a particular isoflavone should be understood as molecules exhibiting any one of more of the functional activities of the isoflavone and may be derived from any source such as being chemically synthesised or identified via screening processes such as natural product screening.

The term “alkyl” is taken to include straight chain, branched chain and cyclic (in the case of 5 carbons or greater) saturated alkyl groups of 1 to 10 carbon atoms, preferably from 1 to 6 carbon atoms, such as methyl, ethyl, propyl, isopropyl, butyl, isobutyl, secbutyl, tertiary butyl, pentyl, cyclopentyl, and the like. The alkyl group is more preferably methyl, ethyl, propyl or isopropyl. The alkyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “alkenyl” is taken to include straight chain, branched chain and cyclic (in the case of 5 carbons or greater) hydrocarbons of 2 to 10 carbon atoms, preferably 2 to 6 carbon atoms, with at lease one double bond such as ethenyl, 1-propenyl, 2-propenyl, 1-butenyl, 2-butenyl, 2-methyl-1-propenyl, 2-methyl-2-propenyl, and the like. The alkenyl group is more preferably ethenyl, 1-propenyl or 2-propenyl. The alkenyl groups may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “alkynyl” is taken to include both straight chain and branched chain hydrocarbons of 2 to 10 carbon atoms, preferably 2 to 6 carbon atoms, with at least one triple bond such as ethynyl, 1-propynyl, 2-propynyl, 1-butynyl, 2-butynyl, and the like. The alkynyl group is more preferably ethynyl, 1-propynyl or 2-propynyl. The alkynyl group may optionally be substituted by one or more of fluorine, chlorine, bromine, iodine, carboxyl, C₁-C₄-alkoxycarbonyl, C₁-C4-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “aryl” is taken to include phenyl, biphenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy or halo.

The term “heteroaryl” is taken to include five-membered and six-membered rings which include at least one oxygen, sulfur or nitrogen in the ring, which rings may be optionally fused to other aryl or heteroaryl rings including but not limited to furyl, pyridyl, pyrimidyl, thienyl, imidazolyl, tetrazolyl, pyrazinyl, benzofuranyl, benzothiophenyl, quinolyl, isopuinolyl, purinyl, morpholinyl, oxazolyl, thiazolyl, pyrrolyl, xanthinyl, purine, thymine, cytosine, uracil, and isoxazolyl. The heteroaromatic group can be optionally substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl. The heteroaromatic can be partially or totally hydrogenated as desired.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” or “prodrug” refers to a derivative of the active compound that upon administration to the recipient, is capable of providing directly or indirectly, the parent compound or metabolite, or that exhibits activity itself.

As used herein, the terms “treatment”, “prophylaxis” or “prevention”, “amelioration” and the like are to be considered in their broadest context. In particular, the term “treatment” does not necessarily imply that an animal is treated until total recovery. Accordingly, “treatment” includes amelioration of the symptoms or severity of a particular condition or preventing or otherwise reducing the risk of developing a particular condition.

The amount of one or more compounds of formula I which is required in a therapeutic treatment according to the invention will depend upon a number of factors, which include the specific application, the nature of the particular compound used, the condition being treated, the mode of administration and the condition of the patient. Compounds of formula I may be administered in a manner and amount as is conventionally practised. See, for example, Goodman and Gilman, et al. (1995). The specific dosage utilised will depend upon the condition being treated, the state of the subject, the route of administration and other well known factors as indicated above. In general, a daily dose per patient may be in the range of 0.1 mg to 5 g; typically from 0.5 mg to 1 g; preferably from 50 mg to 200 mg. The length of dosing may range from a single dose given once every day or two, to twice or thrice daily doses given over the course of from a week to many months to many years as required, depending on the severity of the condition to be treated or alleviated. It will be further understood that for any particular subject, specific dosage regimens should be adjust over time according to the individual need and the professional judgment of the person administering or supervising the administration of the compositions. Relatively short term treatments with the active compounds can be used to cause stabilisation or shrinkage of coronary artery disease lesions that cannot be treated either by angioplasty or surgery. Longer term treatments can be employed to prevent the development of advanced lesions in high-risk patients.

The production of pharmaceutical compositions for the treatment of the therapeutic indications herein described are typically prepared by admixture of the compounds of the invention (for convenience hereafter referred to as the “active compounds”) with one or more pharmaceutically or veterinary acceptable carriers and/or excipients as are well known in the art.

The carrier must, of course, be acceptable in the sense of being compatible with any other ingredients in the formulation and must not be deleterious to the subject. The carrier or excipient may be a solid or a liquid, or both, and is preferably formulated with the compound as a unit-dose, for example, a tablet, which may contain up to 100% by weight of the active compound, preferably from 0.5% to 59% by weight of the active compound. One or more active compounds may be incorporated in the formulations of the invention, which may be prepared by any of the well known techniques of pharmacy consisting essentially of admixing the components, optionally including one or more accessory ingredients. The preferred concentration of active compound in the drug composition will depend on absorption, distribution, inactivation, and excretion rates of the drug as well as other factors known to those of skill in the art.

The formulations of the invention include those suitable for oral, rectal, optical, buccal (for example, sublingual), parenteral (for example, subcutaneous, intramuscular, intradermal, or intravenous) and transdermal administration, although the most suitable route in any given case will depend on the nature and severity of the condition being treated and on the nature of the particular active compound which is being used.

Formulation suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration conveniently comprise sterile aqueous preparations of the active compounds, which preparations are preferably isotonic with the blood of the intended recipient. These preparations are preferably administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound and are administered at a rate of 0.1 ml/minute/kg.

Formulations suitable for rectal administration are preferably presented as unit dose suppositories. These may be prepared by admixing the active compound with one or more conventional solid carriers, for example, cocoa butter, and then shaping the resulting mixture.

Formulations or compositions suitable for topical administration to the skin preferably take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 5% w/w, more particularly from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound. See for example Brown, L., et al. (1998).

Formulations suitable for transdermal administration may also be delivered by iontophoresis (see, for example, Panchagnula R, et al., 2000) and typically take the form of an optionally buffered aqueous solution of the active compound. Suitable formulations comprise citrate or Bis/Tris buffer (pH 6) or ethanol/water and contain from 0.1 M to 0.2 M active ingredient.

Formulations suitable for inhalation may be delivered as a spray composition in the form of a solution, suspension or emulsion. The inhalation spray composition may further comprise a pharmaceutically acceptable propellant such as carbon dioxide or nitrous oxide.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

Therapeutic methods, uses and compositions may be for administration to humans or animals, including mammals such as companion and domestic animals (such as dogs and cats) and livestock animals (such as cattle, sheep, pigs and goats), birds (such as chickens, turkeys, ducks) and the like.

The active compound or pharmaceutically acceptable derivatives prodrugs or salts thereof can also be co-administered with other active materials that do not impair the desired action, or with materials that supplement the desired action, such as antibiotics, antifungals, antiinflammatories, or antiviral compounds. The active agent can comprise two or more isoflavones or derivatives thereof in combination or synergistic mixture. The active compounds can also be administered with lipid lowering agents such as probucol and nicotinic acid; platelet aggregation inhibitors such as aspirin; antithrombotic agents such as coumadin; calcium channel blockers such as verapamil, diltiazem, and nifedipine; angiotensin converting enzyme (ACE) inhibitors such as captopril and enalapril, and β-blockers such as propanolol, terbutalol, and labetalol. The compounds can also be administered in combination with nonsteriodal antiinflammatories such as ibuprofen, indomethacin, aspirin, fenoprofen, mefenamic acid, flufenamic acid and sulindac. The compounds can also be administered with corticosteroids.

The co-administration may be simultaneous or sequential. Simultaneous administration may be effected by the compounds being in the same unit dose, or in individual and discrete unit doses administered at the same or similar time. Sequential administration may be in any order as required and typically will require an ongoing physiological effect of the first or initial active agent to be current when the second or later active agent is administered, especially where a cumulative or synergistic effect is desired.

The isoflavones for use in the present invention may be derived from any number of sources readily identifiable to a person skilled in the art. Preferably, they are obtained in the form of concentrates or extracts from plant sources. Again, those skilled in the art will readily be able to identify suitable plant species, however, for example, plants of particular use in the invention include leguminous plants. More preferably, the isoflavone extract is obtained from chickpea, lentils, beans, red clover or subterranean clover species and the like.

Isoflavone extracts may be prepared by any number of techniques known in the art. For example, suitable isoflavone extracts may be prepared by water/organic solvent extraction from the plant source. It will be appreciated that an isoflavone extract may be prepared from any single tissue of a single species of plant or a combination of two or more different tissues thereof. Similarly, an extract may be prepared from a starting material which contains a heterogeneous mixture of tissues from two or more different species of plant.

Generally, where an isoflavone extract is prepared from plant material, the material may be comminuted or chopped into smaller pieces, partially comminuted or chopped into smaller pieces and contacted with water and an organic solvent, such as a water miscible organic solvent. Alternatively, the plant material is contacted with water and an organic solvent without any pre-treatment. The ratio of water to organic solvent may be generally in the range of 1:10 to 10:1 and may, for example, comprise equal proportions of water and solvent, or from 1% to 30% (v/v) organic solvent. Any organic solvent or a mixture of such solvents may be used. The organic solvent may preferably be a C2-10, more preferably a C1-4 organic solvent (such as methanol, chloroform, ethanol, propanol, propylene glycol, erythrite, butanol, butanediol, acetonitrile, ethylene glycol, ethyl acetate, glycidol, glycerol dihydroxyacetone or acetone). Optionally the water/organic solvent mixture may include an enzyme which cleaves isoflavone glycosides to the aglycone form. The mixture may be vigorously agitated so as to form an emulsion. The temperature of the mix may range, for example, from an ambient temperature to boiling temperature. Exposure time may be between one hour to several weeks. One convenient extraction period is twenty-four hours at 90° C. The extract may be separated from undissolved plant material and the organic solvent removed, such as by distillation, rotary evaporation, or other standard procedures for solvent removal. The resultant extract containing water soluble and non-water soluble components may be dried to give an isoflavone-containing extract, which may be formulated with one or more pharmaceutically acceptable carriers, excipients and/or auxiliaries according to the invention.

An extract made according to the description provided in the previous paragraphs may contain small amounts of oil which include isoflavones in their aglycone form (referred to herein as isoflavones). This isoflavone enriched oil, may be subject to HPLC to adjust the isoflavone ratios, or, if it is at the desired isoflavone ratio, may be dried, for example in the presence of silica, and be formulated with one or more carriers, excipients and/or auxiliaries to give an isoflavone containing extract. Alternatively, the isoflavones contained in said small amounts of oil may be further concentrated by addition to the oil of a non-water soluble organic solvent such as hexane, heptane, octane acetone or a mixture of one or more of such solvents. One example is 80% hexane, 20% acetone w/w having high solubility for oils but low solubility for isoflavones. The oil readily partitions into the organic solvent, and an enriched isoflavone containing extract falls out of solution. The recovered extract may be dried, for example in an oven at 50° C. to about 120° C., and formulated with one or more pharmaceutically acceptable carriers, excipients and/or auxiliaries.

It will be appreciated that the present invention also contemplates the production of suitable isoflavones, functional derivatives, equivalents or analogues thereof, by established synthetic techniques well known in the art. See, for example, Chang et al. (1994) which discloses methods appropriate for the synthesis of various isoflavones.

Other suitable methods may be found in, for example, published International Patent Applications WO 98/08503 and WO 00/49009, and references cited therein, which are incorporated herein in their entirety by reference.

The invention is further illustrated by the following non-limiting Examples and accompanying drawings.

### EXAMPLE 1

### Anti-Vasoconstrictor Effects and Protection from Oxidised LDL Damage

The elasticity and vascular response of blood vessels are important factors contributing to cardiovascular risk. Arteries are responsive to the differential stimuli of stress versus normal physiological conditions is indicative of a healthy circulatory system. Cholesterol accumulation and age contribute to a poorly responsive vessel. Thus, experiments were performed in which the ability of isoflavones to produce vasodilation and to protect vessels from oxidised LDL damage was investigated. Furthermore, the importance of the endothelium in these effects was explored.

Methods

Isolation of rat thoracic aorta

Male Sprague-Dawley rats (250±50 g) were gassed with 80% CO₂ and 20% O₂ until killed. The thoracic aorta was excised and quickly placed in ice-cold Krebs modified solution composed of NaCl 199 mM, HCl 4.7 mM, MgSO₄ 7H2O 1.17 mM, NaHCO₃ 25 mM, KH₂PO₄ 1.18 mM, CaCl₂ 2.5 mM, glucose 11 mM and EDTA 0.03 mM. It was then trimmed of fat and connective tissue and cut into 2-3 mm wide rings. Each ring was mounted on two parallel stainless steel hooks in a single water-jacketed 3 ml organ bath containing modified Krebs solution maintained at 37° C. and bubbled with 95% O₂ plus 5% CO₂. The lower hook was attached to a movable support leg and the upper hook to an FTO3 force transducer (Grass Scientific Instruments, Mitutoyo, Japan) for isometric recordings. Changes in force were amplified (Quadbridge amplifier, Scientific Concepts Inc., Victoria, Australia) and recorded on a MacLab 8E data acquisition system (Adinstruments Pty Ltd., NSW, Australia) linked to an Apple computer (Apple Computer Inc, Cupertina, Calif.). Rings were set at an initial tension of 2 g and allowed to equilibrate for 30 minutes after which time they were re-set to 2 g tension before the commencement of each experimental protocol.

Protocol 1: The Effect of Isoflavone Metabolites on Contractile Curves to Noradrenaline

Full concentration-contractile curves were obtained to noradrenaline (0.1 nM-10 mM) in the absence and presence of β-estradiol (1 μg/ml); Cpd. 5 (0.1 and 1 μg/ml); Cpd. 7 (0.1 and 1 μg/ml); Cpd. 8 (0.1 and 1 μg/ml) and Cpd. 12 (1 μg/ml) and the vehicle equi-volume DMSO. Only one compound at any one concentration was tested on any one ring from any one animal.

Protocol 2a: The Vasodilatory Capacity of Isoflavone Metabolite Derivatives

A full concentration response to noradrenaline was obtained. From this the concentration producing a submaximal contraction of approximately 80% was selected (0.03-0.3 μM). The ring was then constricted with this submaximal concentration and allowed to plateau before full concentration-relaxation curves were obtained to β-estradiol and Cpds. 5, 7, 8 and 12 and the vehicle DMSO (at equi-volume). Only one compound was studied with any one ring from any one animal.

Protocol 2b. The Vasodilatory Capacity of Isoflavone Metabolite Derivatives: Mechanism of Action

Full relaxation curves were obtained to Cpds. 5, 7, 8 and 12 and β-estradiol in the absence and presence of the endothelium (rings were denuded of endothelium by gentle rotation of the lumen against a rough surface), the nitric oxide synthase inhibitor nitro-L-arginine (NOLA 10 μM; with the exception of Cpd. 8, due to limited supply of the compound), KCl 40 mM (to inhibit endothelium derived hyperpolarising factor), the cyclo-oxygenase inhibitor indomethacin (10 μM) and the soluble guanylate cyclase inhibitor 1H-[1,2,4]oxadiazolo[4,3-a]quinoxalin-1-one (ODQ; 10 μM). Only one compound with only one intervention was tested on any one ring from any one animal.

Protocol 3. Protective Effect of Isoflavone Metabolite Derivatives Against Endothelium Damage Induced by Oxidised Low-Denisity Lipoprotein

Full concentration response curves to phenylephrine were constructed from which the concentration giving a submaximal contraction of approximately 80% was selected (usually 0.3 μM). Tissues were then constricted with phenylephrine at the selected concentration and allowed to plateau. Full concentration dilatory curves were then constructed to acetylcholine. Following this, 0.1% antifoam B (SIGMA), demonstrated in preliminary experiments not to affect responses to acetylcholine, was added to the bath. Full concentration response curves to acetylcholine were then repeated following a 1 hour incubation with oxidised-low density lipoprotein (ox-LDL: 0.3 mg protein/ml) in the absence and added presence of β-estradiol (10 pg/ml), Cpd. 5 (300 ng/ml), Cpd. 7 (1 μg/ml), Cpd. 8 (3 μg/ml) or Cpd. 12 (3 μg/ml). The concentrations were chosen based on neg log EC₄₅-EC₅₀ from the experiments performed in Protocol 2a.

Preparation of Low Density Lipoprotein

Human plasma was obtained from the blood bank. Individual lipoprotein fractions were obtained using discontinuous density gradient ultracentrifugation on a Beckman vertical rotor (70 Ti) with a Beckman centrifuge. In short, 0.01 8g of KBr was added to each ml of plasma, this was transferred to quick seal tubes and spun at 65,000 rpm, 4° C. overnight. The top (VLDL) fraction was then removed and 0.064 g of KBr added to each ml of the bottom section. This was transferred to quick-seal tubes and spun at 65000 rpm, 4° C. overnight. The top (LDL) fraction was transferred to quick seal tubes and topped up with dl 0.063 solution and spun. The top LDL fraction was collected following each spin and dialysed against 0.05M NH₄HCO₃ pH 8.0 over 4 days with changes of solution each day. The protein content of the final LDL fraction obtained was determined by the simplified protein assay method of Lowry. LDL was oxidised by a 2 hr incubation with CuSO₄ 5 μM.

Data Presentation and Statistical Analysis

Contractile responses are expressed in g tension (mean+standard error of the mean). Dilatory responses are expressed as a percentage of the contractile force generated by the pre-constricting agent used. Individual concentration-response curves for noradrenaline and acetylcholine±the interventions used were fitted to a logistic equation of the form
E=MA^{p}/(A^{p}XK^{p})
where E is the response, M is the maximum response and K is the concentration eliciting 50% of the maximum response (ie neg log EC₅₀).

Results were analysed by 2 way repeated measure analysis of variance where appropriate followed by post-hoc t-tests with the appropriate corrections using Sigmastat Statistical software (Jandel Scientific, San Rafael, Calif.) which inherently tests data for normality prior to performing parametric analysis.

Drugs and Solutions

1,3,5[10]-estratriene-3,17β-diol (17β-estradiol, SIGMA), Cpd. 5, Cpd. 7, Cpd. 8 and Cpd. 12 (manufactured by Novogen Ltd) were dissolved in DMSO and diluted to the required concentrations in Krebs. N^{ω}-nitro-L-arginine (SIGMA), indomethacin (SIGMA), norepinephrine bitartrate salt (SIGMA), acetylcholine chloride (SIGMA) and 1H-[1,2,4]oxadiazolo[4,3-a]quinoxalin-1-one (SIGMA) were dissolved to the manufacturers specifications.

Results

Protocol 1:The Effect of Isoflavone Metabolites on Contractile Curves to Noradrenaline

Table 1 lists the maximal force obtained to noradrenaline in the absence and presence of β-estradiol, the synthetic isoflavone derivatives (Cpds. 5, 7, 8 and 12) and the used.

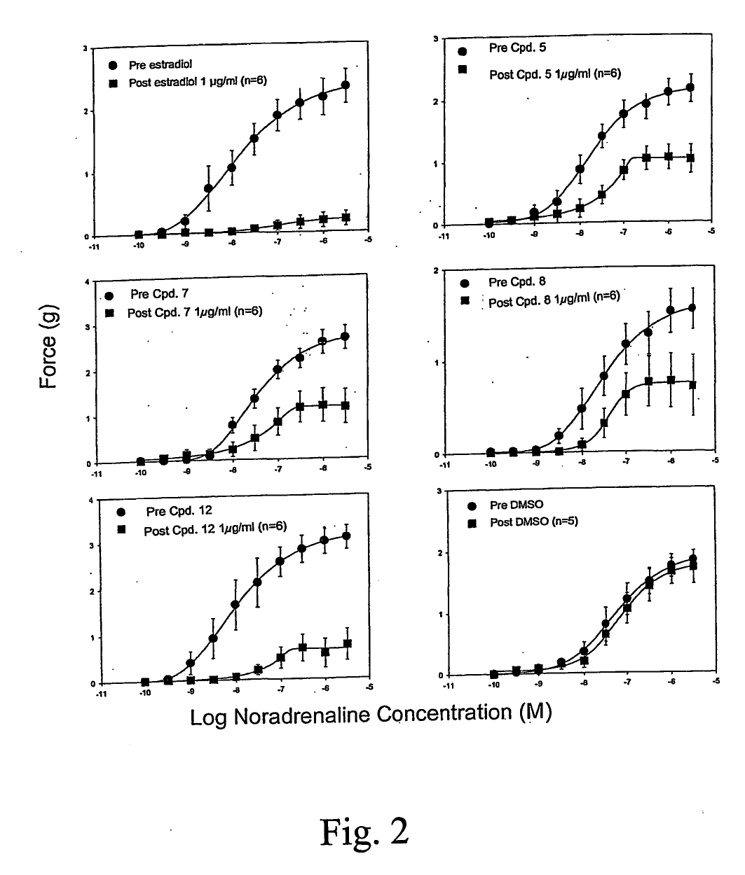

FIG. 1 shows in graphical form the effects on responses to noradrenaline for various compounds when used at 1 μg/ml. FIG. 2 shows full concentration-contractile responses to noradrenaline in the absence (pre-compound) and presence following a 30 min incubation (post-compound).

All the active compounds studied had a significant antagonistic effect on noradrenaline-induced contraction. To obtain an index of the order of potency of the compounds studied, the difference in maximal response to noradrenaline (the maximal response in the absence of the compounds minus the maximal response in the presence of compounds) was calculated for each compound at 1 μg/ml (Table 1). From this it was clear that Cpd. 12 was comparable to β-estradiol in antagonising the contractile response to noradrenaline, while Cpd. 5, Cpd. 7 and Cpd. 8 were active but less potent than β-estradiol.

TABLE 1
Maximal force (y max) generated by noradrenaline in the absence or
presence of β-estradiol and Cpds. 5, 7, 8 and 12

| | | y max (g tension) | | |
|---|---|---|---|---|
| | | Noradrenaline | Noradrenaline + Test | |
| | | alone (A) | compound (B) | Difference (A − B) |
| B-estradiol | 1 μg/ml | 2.32 ± 0.27 | 0.22 ± 0.12* | 2.1 ± 0.31 |
| Cpd. 5 | 0.1 μ/ml | 2.30 ± 0.22 | 1.50 ± 0.32* | 0.81 ± 0.25 |
| | 1 μg/ml | 2.15 ± 0.22 | 1.13 ± 0.20* | 1.02 ± 0.24 |
| Cpd. 7 | 0.1 μg/ml | 1.66 ± 0.14 | 1.41 ± 0.13 | 0.25 ± 0.25 |
| | 1 μg/ml | 2.64 ± 0.26 | 1.26 ± 0.34* | 1.38 ± 0.27 |
| Cpd. 8 | 0.1 μg/ml | 1.90 ± 0.09 | 1.34 ± 1.20* | 0.56 ± 0.15 |
| | 1 μg/ml | 1.55 ± 0.22 | 0.86 ± 0.31* | 0.69 ± 0.28 |
| Cpd. 12 | 1 μg/ml | 3.07 ± 0.26 | 0.80 ± 0.36* | 2.27 ± 0.68 |
| Ethanol | | 0.89 ± 0.36 | 1.51 ± 0.29 | 0.38 ± 0.57 |
| DMSO | | 1.82 ± 0.16 | 1.81 ± 1.23 | 0.01 ± 0.28 |

*P ≦ 0.05 B compared with A

Protocol 2a. The Vasodilatory Capacity of Isoflavone Metabolites: Mechanisms of Action

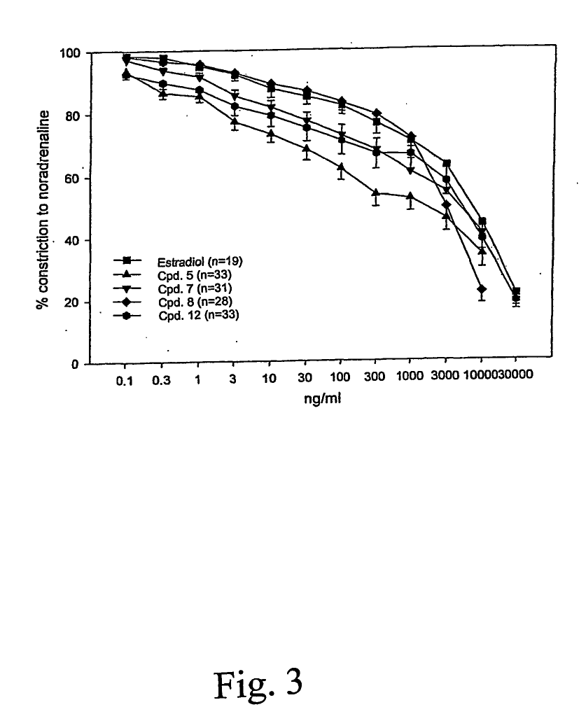

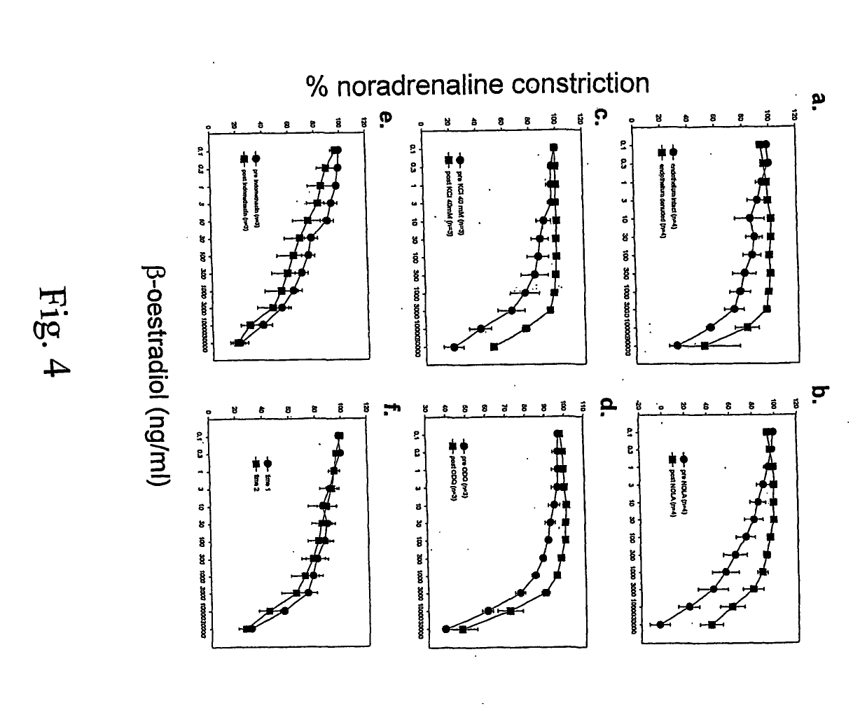

FIG. 3 depicts the concentration-dilatory effects of the compounds studied. Concentration-dilatation curves obtained for 17β-estradiol, and Cpds. 5, 7, 8 and 12 with rat isolated aortic rings pre-constricted with a submaximal concentration of noradrenaline. All values are mean±standard error of the mean. All four compounds were at least as potent as β-estradiol in their vasodilatory capacity. The mechanism by which these compounds exerted the dilatory effect was further examined using specific antagonists. β-estradiol: FIG. 4 depicts concentration-dilatation curves obtained for 17β-estradiol with rat isolated aortic rings pre-constricted with a submaximal concentration of noradrenaline. These were performed in the absence and presence of (a) intact endothelium, (b) nitric oxide synthase inhibitor N^{ω}-nitro-L-arginine (NOLA 10 μM); (c) 40 mM KCl, (d) soluble guanylate cyclase inhibitor 1H-[1,2,4]oxadiazolo[4,3-a]quinoxalin-1-one (ODQ 10 μM), (e) cyclo-oxygenase inhibitor indomethacin (10 μM) and (f) represents a time (30 min) control. n=number of experiments.

Vasodilatory responses to β-estradiol were inhibited by removal of the endothelium (FIG. 3a), incubation with nitro-L-arginine (FIG. 4b), high KCl levels (FIG. 4c) and ODQ (FIG. 4d). Indomethacin did not influence the dilatory effects of this steroid (FIG. 4e) and no time dependent changes were observed (FIG. 4f).

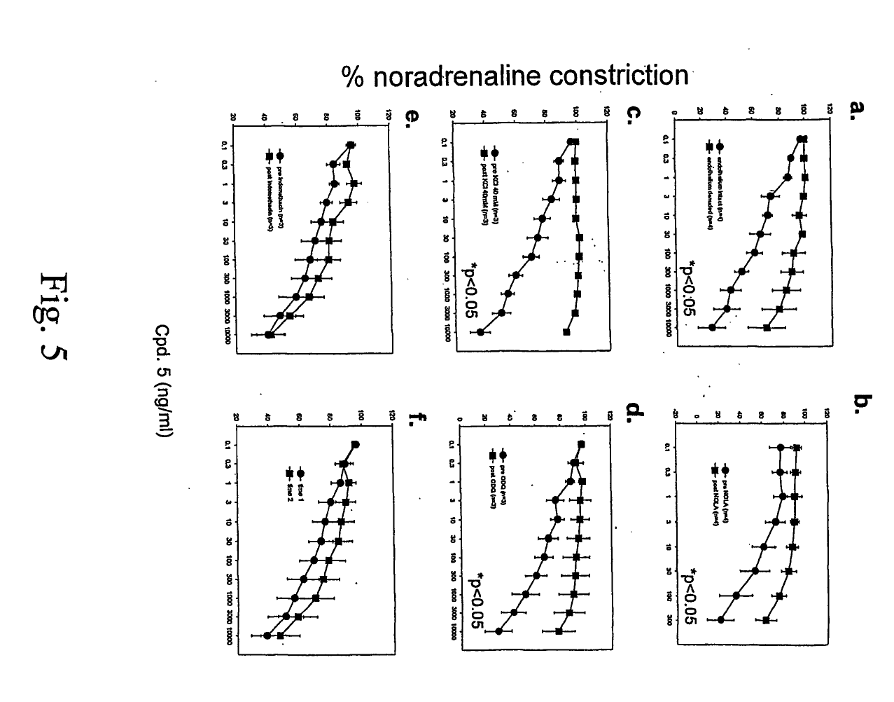

Cpd. 5: FIG. 5 depicts concentration-dilatation curves obtained for Cpd. 5 with rat isolated aortic rings pre-constricted with a submaximal concentration of noradrenaline. These were performed in the absence and presence of (a) intact endothelium, (b) nitric oxide synthase inhibitor N^{ω}-nitro-L-arginine (NOLA 10 μM); (c) 40 mM KCl, (d) soluble guanylate cyclase inhibitor 1H-[1,2,4]oxadiazolo[4,3-a]quinoxalin-1-one (ODQ 10 μM), (e) cyclo-oxygenase inhibitor indomethacin (10 μM) and (f) represents a time (30 min) control. n=number of experiments

Vasodilatory responses to Cpd. 5 were inhibited by removal of the endothelium (FIG. 5a), incubation with nitro-L-arginine (FIG. 5b), high KCl levels (FIG. 5c) and ODQ (FIG. 5d). Indomethacin did not influence the dilatory effects of this steroid (FIG. 5e) and no time dependent changes were observed (FIG. 5f).

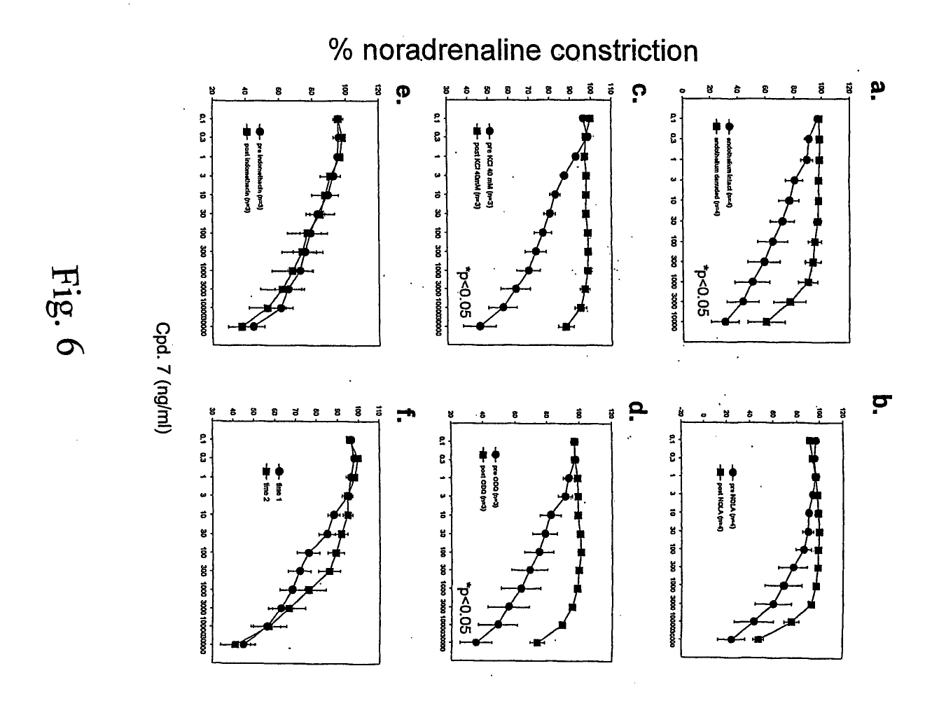

Cpd. 7: FIG. 6 depicts concentration-dilatation curves obtained for Cpd. 7 with rat isolated aortic rings pre-constricted with a submaximal concentration of noradrenaline. These were performed in the absence and presence of (a) intact endothelium, (b) nitric oxide synthase inhibitor N^{ω}-nitro-L-arginine (NOLA 10 μM); (c) 40 mM KCl, (d) soluble guanylate cyclase inhibitor 1H-[1,2,4]oxadiazolo[4,3-a]quinoxalin-1-one (ODQ 10 μM), (e) cyclo-oxygenase inhibitor indomethacin (10 μM) and (f) represents a time (30 min) control. n=number of experiments

Vasodilatory responses to Cpd. 7 were inhibited by removal of the endothelium (FIG. 6a), high KCl levels (FIG. 6c) and ODQ (FIG. 6c). There was a trend towards inhibition of these responses by NOLA but this was not statistically significant. Indomethacin did not influence the dilatory effects of this steroid (FIG. 6d) and no time dependent changes were observed (FIG. 6e).

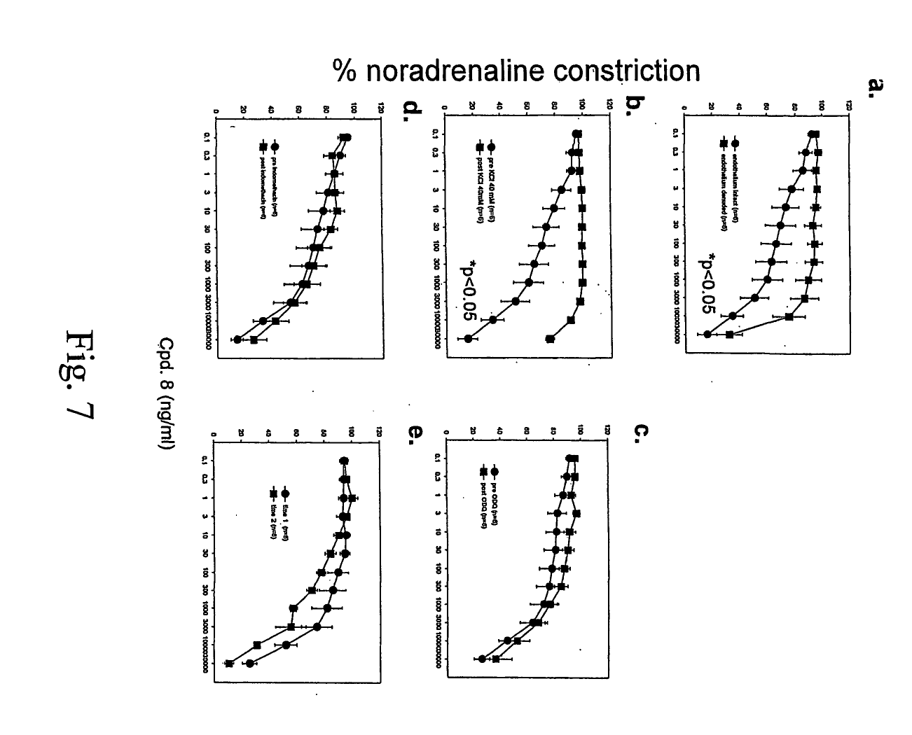

Cpd. 8: FIG. 7 depicts concentration-dilatation curves obtained for Cpd. 8 with rat isolated aortic rings pre-constricted with a submaximal concentration of noradrenaline. These were performed in the absence and presence of (a) intact endothelium, (b) 40 mM KCl, (c) soluble guanylate cyclase inhibitor 1H-[1,2,4]oxadiazolo[4,3-a]quinoxalin-1-one (ODQ 10 μM), (d) cyclo-oxygenase inhibitor indomethacin (10 μM) and (e) represents a time (30 min) control. n=number of experiments

Vasodilatory responses to Cpd. 8 were inhibited by removal of the endothelium (FIG. 7a), high KCl levels (FIG. 7b) but not by ODQ (FIG. 7c). Indomethacin potentiated the inhibitory effects of Cpd. 8 (FIG. 7d). No time dependent changes were observed (FIG. 7e).

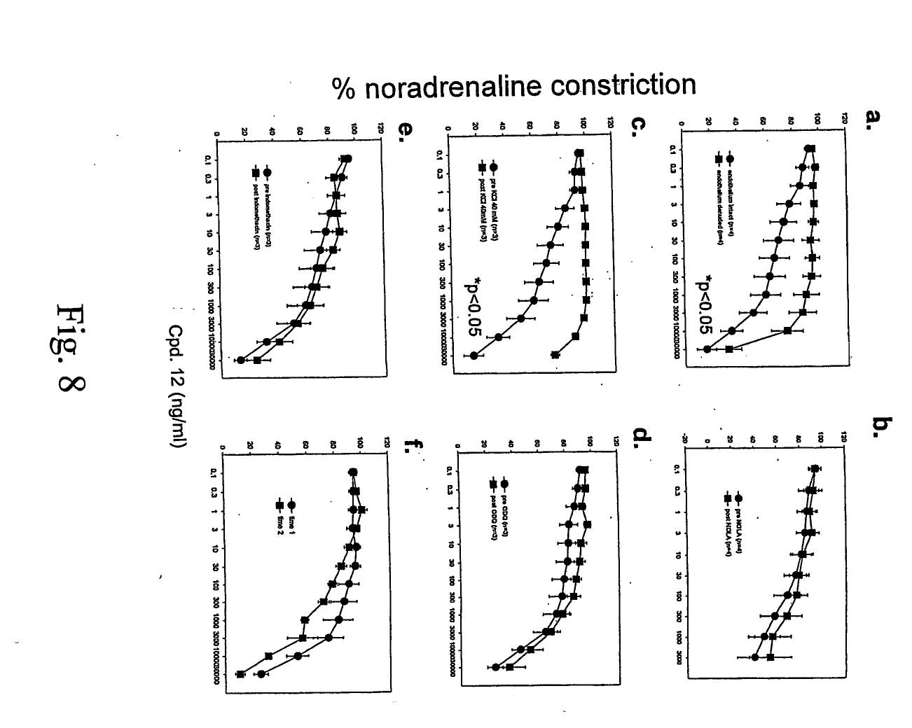

Cpd. 12: FIG. 8 depicts concentration-dilatation curves obtained for Cpd. 12 with rat isolated aortic rings pre-constricted with a submaximal concentration of noradrenaline. These were performed in the absence and presence of (a) intact endothelium, (b) nitric oxide synthase inhibitor N^{ω}-nitro-L-arginine (NOLA 10 μM); (c) 40 mM KCl, (d) soluble guanylate cyclase inhibitor 1H-[1,2,4]oxadiazolo[4,3-a]quinoxalin-1-one (ODQ 10 μM), (e) cyclo-oxygenase inhibitor indomethacin (10 μM) and (f) represents a time (30 min) control. n=number of experiments

The vasodilatory effects of Cpd. 12 were inhibited by removal of the endothelium (FIG. 8a) and high KCl levels (FIG. 8c) but not by NOLA (FIG. 8b) or ODQ (FIG. 8d) nor indomethacin (FIG. 8e). No time dependent changes were observed (FIG. 8e).

Protocol 3. Protective Effect of Isoflavone Metabolites Against Endothelium Damage Induced by Oxidised Low-Density Lipoprotein

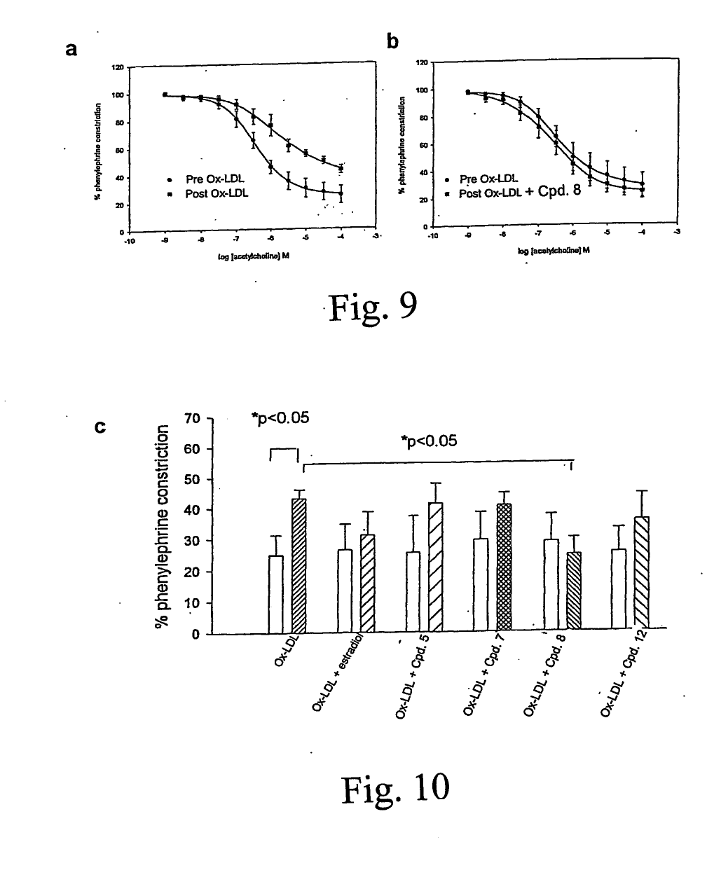

FIG. 9 depicts fall concentration-dilatation curves to acetylcholine obtained with rat isolated aortic rings pre-constricted with a submaximal concentration of phenylephrine in the absence and presence following a 30 min incubation with (a) oxidised low density lipoprotein (ox-LDL 0.3 mg protein/ml); (b) Ox-LDL+Cpd. 8 (3 μg/ml). FIG. 10 represents histograms depicting maximal dilatation obtained to acetylcholine with rat isolated aortic rings pre-constricted with a submaximal concentration of phenylephrine in the absence and presence of sole incubation with ox-LDL and co-incubation of oxLDL+17β-estradiol or Cpds. 5, 7, 8 or 12. n=number of experiments.

Responses to acetylcholine were significantly attenuated by incubation with ox-LDL (FIG. 9a). Co-incubation of ox-LDL with Cpd. 8 diminished the inhibitory effect of ox-LDL (FIG. 9b).

Further, the maximal response obtained to acetylcholine in the presence of co-incubation with this compound was significantly less than with sole incubation with ox-LDL (FIG. 10). Co-incubation of ox-LDL with β-estradiol and the other compounds diminished the effect of ox-LDL such that the significant difference between the maximal dilatation obtained before and after ox-LDL incubation was no longer apparent. However maximal dilatation to acetylcholine obtained in the presence of the co-incubation was no different to that obtained in the presence of only ox-LDL (FIG. 10).

Discussion

These studies demonstrate that the isoflavone compounds and derivatives (a) inhibit vasoconstrictive responses to noradrenaline (b) are vasodilatory and (c) that Cpd. 8 significantly protects against endothelium damage by oxidized LDL. The in vitro vascular profile of some of these compounds is comparable to, and in some cases more effective than, that of the ovarian steroid 17β-estradiol. This suggests that these compounds may be responsible for the cardioprotective effects attributed to high isoflavone diets but more importantly that these compounds may be useful as potential cardioprotective therapeutic agents especially if administered in doses greater that those achieved from normal dietary isoflavone intake.

Cpds. 5, 7, 8 and 12 were all able to antagonize the contractile effects of noradrenaline but to differing degrees. While Cpd. 12 was most effective and comparable to 170-estradiol, Cpd. 5 and Cpd. 7 appeared equipotent but less potent than Cpd. 12. Cpd. 8 was the least potent. The antagonising action of Cpds. 5, 7 and 8 were dose dependent but dose dependency of Cpd. 12 and β-estradiol were not examined. At equi-volume, the vehicle (DMSO) was ineffective at inhibiting responses to noradrenaline.

While the vasodilatory effects of the isoflavone metabolite synthetic derivatives were at least as potent as, if not more potent than, 17β-estradiol, the mechanism of action differed from that of 17β-estradiol in that the dilatory action of all four compounds diminished upon removal of the endothelium.

None of the compounds were affected by indomethacin suggesting that prostacyclin is unlikely to play a substantial role in their vasodilatory capacity. Indomethacin did however potentiate the effects of Cpd. 8 such that an increase in vasodilatory capacity was observed suggesting that Cpd. 8 increased the release of a vasoconstrictory prostanoid, such as thromboxane, the removal of which led to the increased dilatation observed. The dilatory responses of both Cpd. 5 and Cpd. 7 were inhibited by high levels of KCl, ODQ and NOLA. The dilatory action of both these compounds can therefore be associated with the release of an endothelium derived relaxing factor, probably nitric oxide activating soluble guanylate cyclase activity.

The inhibition by KCl suggests that an endothelium derived hyperpolarising factor (EDHF) is also released. It is recognised that there are limitations to the use of KCl in isolating EDHF responses, and that further investigation is warranted with the use of specific K⁺ channel inhibitors such as apamin and charybdotoxin. Given the limitations, in the current study, there is no evidence to suggest that the hyperpolaring factor released by Cpd. 5 and Cpd. 7 is not nitric oxide.

The dilatory effects of Cpd. 8 and Cpd. 12, on the other hand, can be totally attributed to EDHF which in this instance is clearly not nitric oxide and not activated via soluble guanylate cyclase. It would thus appear that the mechanism of action by which these four compounds cause vasodilatation differs not only from the ovarian steroid β-estradiol and the parent compounds, but also from each other.

In the final series of experiments the protective effect of these compounds against endothelium damage induced by oxidised LDL was examined. Oxidised LDL inhibited the vasodilatory capacity of the endothelium-dependent vasodilator acetylcholine. While all the compounds studied diminished the responses to acetylcholine due to ox-LDL, Cpd. 8 was the only compound which could be demonstrated to have a significant effect on responses to acetylcholine in direct comparison with sole incubation with ox-LDL. That 17β-estradiol can protect against endothelial damage by ox-LDL has previously been demonstrated. From the current data, it would appear that the protective effect of Cpd. 8 in this context is at least 10 times more potent than 17β-estradiol. Since Cpd. 8 is not the most potent compound in the other protocols, ie. in either antagonising noradrenaline nor in its direct vasodilatory capacity, the mechanism of the cardioprotective action of this compound is likely to be different to the others tested and may lie in its anti-oxidant capacity.

These results show that Cpds. 5, 7, 8 12 have potent vascular regulatory capacity. Thus isoflavones and derivatives thereof are capable of antagonising contractile activity (Cpd. 12>Cpd. 5=Cpd. 7>Cpd. 8), antagonising direct vasodilatation, and protecting against endothelium damage by oxidised low density lipoprotein (Cpd. 8 in this context is at least 10 times more potent than 17β-estradiol). While comparable to the ovarian steroid 17β-estradiol, their activities also appear to be unique in their mechanism of action. Thus the isoflavones and derivatives show potential for use as cardioprotective therapeutics, and in particular for use in the treatment of restenosis and other types of accelerated intimal thickening.

### EXAMPLE 2

### Vascular Smooth Muscle Cell Effects

Vascular smooth muscle cell proliferation is an important step in the atherosclerotic process and is inhibited by estrogens. In order to investigate the atheroprotective properties of the isoflavone compounds and derivatives, experiments were performed to examine the effects of the selected synthetic derivatives of isoflavone metabolites, Cpds. 5, 7, 8 and 12, on simple markers of DNA synthesis and cell proliferation, namely [3H]-thymidine incorporation and cell numbers. In view of the promising results obtained for Cpd. 7, the signalling pathways mediating these activities were examined by determining the effect of Cpd. 7 on the mitogen-activated protein (MAP) kinases (Erk, JUNK, p38).

Methods

Cell Preparation

Vascular smooth muscle cells (VSMC) derived from female internal mammary artery (IMA) were cultured in 10% FBS-DMEM. Cells were grown to confluence in LG (glucose 5 mM)-medium then trypsinized, diluted with 10% FBS-LG medium to about 10,000 cells/ml and 1 ml of suspension was) aliquoted into four 24-well plates. Cells were then allowed to grow for 2 days to visual confluence. LG serum-free media was changed for 48 h to synchronize most cells at Go/G₁.

Cell Treatment:

After 24 h of serum-free medium, cells were treated with the compounds (Cpd. 5, Cpd. 7, Cpd. 8 or Cpd. 12) for 4 h and FBS was added (final concentration 2.5%) over night (18-20 hours). 4 wells were allocated for each treatment:

(1) Control: 2.5% FBS

(2) FBS+isoflavone treatment at 0.01 mM

(3) FBS+isoflavone treatment at 0.1 mM

(4) FBS+isoflavone treatment at 1 mM

(5) FBS+isoflavone treatment at 10 mM

(6) FBS+isoflavone treatment at 100 mM

Cell proliferation was induced by a variety of growth factors such as serum and platelet derived growth factor (PDGF).

Thymidine Incorporation Assay:

Media was changed to LG serum-free DMEM and cells were treated with [³H]-thymidine at 1 μCi/well for 3 hours. Cells were washed twice with ice cold Dulbecco's PBS. Next, ice cold 0.2M HClO₄ (1 ml/well) was added and cells were incubated on ice for 30 min. Cells were then washed three times with 0.5 ml/well of ice cold 0.2M HClO₄. Media (0.5 ml/well of 0.2 M NaOH) was replaced and cells were incubate at 37° C. After 1 hour, cells were treated with acetic acid (6%, 0.2 ml/well), transferred to scintillation vials with 3 ml scintillation liquid (Instagel) and counted for 2 min per vial.

Results

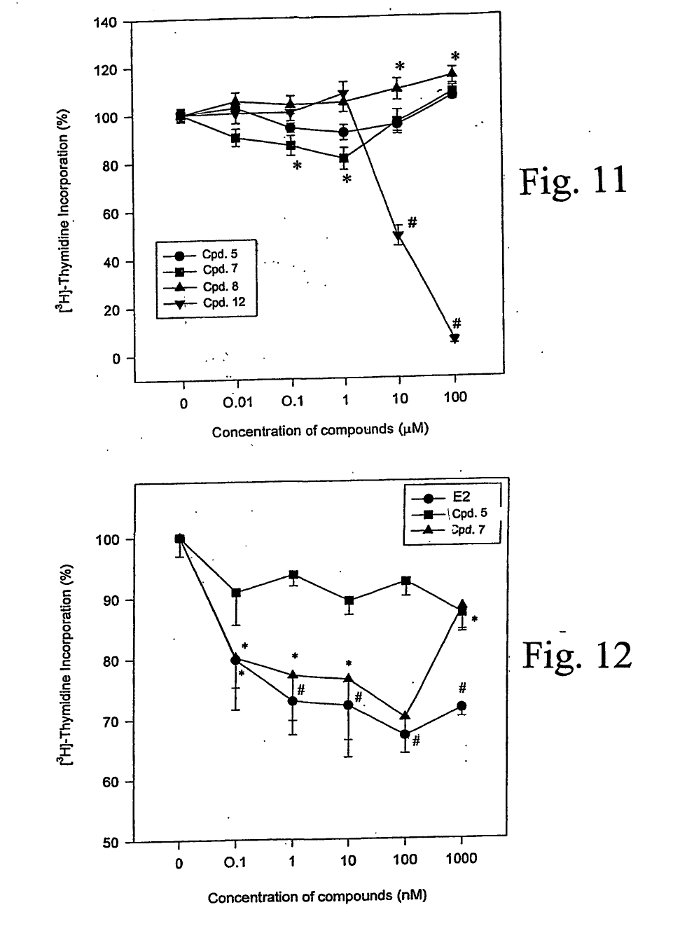

In preliminary experiments it was found that Cpd. 7, at concentrations as low as 0.1 mM, significantly inhibited proliferation of the IMA-derived VSMC cells (n=3)*P<0.05, compared to control (0); # cell damage (see FIG. 11). Interestingly, the effect was only observed at low concentrations (up to 1 mM) and a relative increase in proliferation was measured at 10 mM and 100 mM (FIG. 11). Cpd. 12, although it did not appear to inhibit cellular proliferation, caused significant cell damage at high concentrations (10 mM and 100 mM) (FIG. 11). Studies are currently under way to examine this effect in more detail and determine whether it is due to apoptosis. It was interesting to find that Cpd. 8, at high concentrations (10 mM and 100 mM), caused a significant increase in proliferation (FIG. 11). Cpd. 5 was found to be relatively inactive.

The effect of Cpd. 5 and Cpd. 7 was tested further in IMA-derived human VSMC primed with PDGF BB instead of serum. Again it was found that Cpd. 7, at concentrations as low as 0.1 nM, inhibited proliferation of the IMA cells (n=1)*P<0.005 vs control (0); # P<0.01 vs control (0). (FIG. 12). This effect was equivalent to that observed with estradiol. Cpd. 5 had no observed effect on cellular proliferation

In further experiments Cpd. 7 inhibited platelet-derived growth factor (PDGF) BB-induced DNA synthesis, assessed by [3H]-thymidine incorporation, in a dose-dependent manner with 10% inhibition at 1 nmol/L and 17% inhibition at 10 and 100 nmol/L. The inhibition was statistically significant but less compared to the effect of 17β-estradiol (27% at 1 or 10 nmol/L and 33% at 100 nmol/L). ICI 182780, a non-selective estrogen receptor (ER) antagonist (100 nmol/L) completely abolished the inhibitory effect of Cpd. 7 at 1-10 nmol/L) and partly abolished that of Cpd. 7 at 100 nmol/L), indicating ER-mediation. Consistent with our results with thyrnidine incorporation, PDGF BB-induced increases in cell number were partly but significantly reduced by Cpd. 7 (1-100 nmol/L).

To further examine the signalling pathway mediating the effect of this phytoestrogen in VSMC, the effect of Cpd. 7 on mitogen-activated protein (MAP) kinases (Erk, JUNK, p38) was determined by immunoprecipitation analysis and on early growth response genes (Egr-1, Sp-1) by Western blotting. PDGF BB activated Erk and this activation was significantly inhibited by Cpd. 7 in a dose-dependent manner, while JUNK and p38 activity were not affected by either PDGF BB or Cpd. 7, though the cells strongly expressed both proteins as well as Erk. Egr-1 or Sp-1 proteins were not altered by either PDGF-BB or Cpd. 7.

These results show that isoflavones and derivatives thereof, and in particular Cpd. 7; inhibits cellular proliferation of human vascular smooth muscle cells, and also inhibits PDGF-induced Erk activation in human vascular smooth muscle cells. This activity show the potential for isoflavones and derivatives thereof to prevent the development and progression of atherosclerotic lesions, and in particular accelerated stenosis, providing a potential benefit in vascular protection.

### EXAMPLE 3

### Antioxidant Activity

Oxidation is a process which plays a role in many major disease processes. In the context of cardiovascular disease one of the major contributors to development of atherosclerosis is the oxidation of LDL cholesterol in the arterial wall, leading to an inflammatory lesion. If left unchecked the inflammatory process proceeds until the lesion causes vascular obstruction and infarct, and presents a particularly important problems in the accelerated intimal thickening of restenosis. In view of the potential cardiovascular benefits of anti-oxidant activity, selected isoflavone compounds of the invention were subjected to assays to determine their antioxidant activity.

The LDL Antioxidation Test

The LDL antioxidation test measures the ability of a compound to directly scavenge free radicals or to chelate transition metals. Thus in this series of experiments, the isoflavones and their derivatives were tested for their ability to act as free radical scavengers.

Methods

Fresh whole blood was obtained by venipuncture from healthy human volunteers under 25 years of age and supplemented immediately with EDTA (91 mg/ml) and BHT (4.4 mg/ml). LDL was prepared by step-wise ultracentrifugation within a density gradient of 1.02-1.05 g/cm³. EDTA and BHT were present throughout all the steps of the isolation. The EDTA/BHT containing stock solution (15-30 mg LDL/ml) was stored in the dark in a nitrogen atmosphere until use, but never longer than two weeks.

Before oxidation experiments, the LDL stock solution was dialyzed in the 100-fold volume of 0.01 M phosphate buffer pH 7.4, 0.16 M NaCl, 0.1 mg/ml chloroamphenicol, which was made oxygen-free by vacuum degassing following by purging with nitrogen. The buffer was changed four times. This EDTA- and BHT-free LDL stock solution was used for all oxidation studies. The stock solution was stored not longer than 24 hr at 4° C. For performing the oxidation experiments, the EDTA- and BHT-free LDL stock solution was diluted with oxygen-saturated 0.01 M phosphate buffer pH 7.4, 0.16 M NaCl and the oxidation was initiated by the addition of a freshly prepared aqueous CuCl₂ solution. The final conditions were in all experiments: room temperature, 0.25 mg LDL/ml and 1.66 mM CuCl₂ (see Esterbauer et. al., 1989)

The antioxidant activities of the following compounds were tested: daidzein (10 mM), genistein (10 mM), glycitein (10 mM), biochanin (10 mM), formononetin (10 mM), Promensil™ (Novogen) (2.5 mg/ml, 5 mg/ml), Cpd. 5 (10 mM), Cpd. 7 (10 mM), Cpd. 8 (10 mM), Cpd. 12 (10 mM), Cpd. 4 (10 mM) and Cpd. 6 (10 mM). In each experiment, controls were used to compare the relative effectiveness of the Novogen isoflavone derivatives to common and well-known antioxidants such as ascorbate (2.5 mM; Vitamin C).

Results

It was found that at the concentration tested (10 mM), the parent isoflavones showed no ability to scavenge free radicals. On the other hand, Promensil was shown to be a moderate scavenger, increasing the lag time by up to 100% (at 5 mg/ml) when compared to the positive control (see Table 2).

TABLE 2
Antioxidant activity of the isoflavone parent compounds, expressed as
the lag time, relative to the effect produced by ascorbate. Note:
the longer the lag time, the more active the compound as an antioxidant.

| | | |
|---|---|---|
| Conc. | Lag Time (min) | % Increase in Time |

Compound

| | | | |
|---|---|---|---|
| Control | — | 20 | — |
| Ascorbate | 2.5 μM | 50 | 150 |
| Parent Compounds | | | |
| Promensil | 5 μg/ml | 40 | 100 |
| Promensil | 2.5 μg/ml | 30 | 50 |
| Formononetin | 10 μM | 20 | 0 |
| Genistein | 10 μM | 20 | 0 |
| Glycitein | 10 μM | 20 | 0 |
| Biochanin | 10 μM | 20 | 0 |
| Daidzein | 10 μM | 19 | 0 |

When the isoflavone derivatives were tested, it was found that Cpd. 12 (10 mM) and Cpd. 8 (10 mM) were very active scavengers, increasing the lag time by over 600%, when compared to the positive control (see Table 3). Cpd. 7, was found to be significantly less active than Cpd. 8 and Cpd. 12 but still almost as active as ascorbate.

TABLE 3
Antioxidant activity of the isoflavone derivatives, expressed as the
lag time, relative to the effect produced by ascorbate. Note: the longer the
lag time, the more active the compound as an antioxidant.

| | | |
|---|---|---|
| Conc. | Lag Time (min) | % Increase in Time |

Compound

| | | | |
|---|---|---|---|
| Control | — | 20 | — |
| Ascorbate | 2.5 μM | 70 | 250 |
| Derivatives | | | |
| Cpd. 5 | 10 μM | 20 | 0 |
| Cpd. 7 | 10 μM | 50 | 100 |
| Cpd. 8 | 10 μM | >140 | >600 |
| Cpd. 12 | 10 μM | >140 | >600 |

Prevention of Tocopherol-Mediated Peroxidation (Anti-TMP Test)

Studies were carried out to determine whether the isoflavone derivatives can prevent LDL oxidation in the presence of Vitamin E. This test is physiologically important, since Vitamin E (a-tocopherol) is present with LDL in the blood stream, and LDL oxidation is believed to be one of the major factors of the development of atherosclerosis.

Methods

The anti-TMP test has been described in detail by Bowry et al. (1995). The test indirectly assesses the ability of a compound to synergise with a-tocopherol in human LDL undergoing mild and chemically controlled oxidation. Oxidation is measured by the accumulation of cholesterol ester hydroperoxides at a time point corresponding to 20% consumption of endogenous a-tocopherol.

Briefly, ascorbate and ubiquinol-10-free LDL (1 mM in apoB) was supplemented with an aliquot of a stock solution of the compound to be tested. The mixture was incubated at 37° C. for 10 min and subsequently oxidized at 37° C. with a low flux of water-soluble ROO generated from AAPH (4 mM). The time-dependent consumption of LDL a-tocopherol and accumulation of cholesteryllinolate hydroperoxide (Ch18:2-OOH) were monitored by HPLC with electrochemical and post-column chemiluminescence detection, respectively. The effectiveness of an antioxidant, assigned as its anti-TMP index (redox index), was defined as the relative amounts of Ch18:2-OOH formed with versus without the added antioxidant measured after 20% consumption of the endogenous a-tocopherol in the control sample (ie. in the absence in the antioxidant). Active compounds give rise to low redox index. A high redox activity suggests that the compounds are capable of interacting with the a-tocopherol in LDL, perhaps by reducing the a-tocopheroxyl radical. Butylated hydroxytoluene (BHT, 10 mM) was used as a positive control.

Results

It was found that Cpd. 12 (10 mM) has strong LDL protective action (low redox index), preventing LDL oxidation in the presence of Vitamin E. Furthermore, Cpd. 7 and Cpd. 8 showed low levels of activity. Results are summarised in Table 4.

TABLE 4
Antioxidant activity of the isoflavone derivatives, calculated in
the presence of Vitamin E and expressed as Redox Index. Note: low index
values indicate high redox activity (ie.highly active compounds). Results
are expressed as mean ± % RSD; (n = 2-3)

| | Conc. | Lag Time (min) |
|---|---|---|
| Compound | | |
| Control | — | 100 |
| Butylated hydroxytoluene | 10 μM | 6.3 |
| Derivatives | | |
| Cpd. 5 | 10 μM | 62.3 ± 4 |
| Cpd. 7 | 10 μM | 30.4 ± 10 |
| Cpd. 8 | 10 μM | 26.3 ± 17 |
| Cpd. 12 | 10 μM | 4.5 ± 1 |

In addition, a number of parent isoflavones were tested and they were found to have no antioxidant effect. Promensil showed low levels of activity (see Table 5).

TABLE 5
Antioxidant activity of the isoflavone parent compounds,
calculated in the presence of Vitamin E and expressed as redox index.
Note: low index values indicate high redox activity (ie. highly active
compounds). Results are expressed as mean ± % RSD; (n = 2-3)

| | Conc. | Lag Time (min) |
|---|---|---|
| Compound | | |
| Control | — | 100 |
| Butylated hydroxytoluene | 10 μM | 6.3 |
| Parent Compounds | | |
| Promensil | 0.04 mg/ml | 23.3 ± 2 |
| Formononetin | 10 μM | 73.5 ± 3 |
| Biochanin | 10 μM | 75.3 ± 9 |
| Daidzein | 10 μM | 96.6 ± 15 |
| Glycitein | 10 μM | 115.1 ± 20 |
| Genistein | 10 μM | 115.6 ± 12 |

Synergism With A-Tocopherol (TRAA Test)

The aim of this study was to assesses the ability of the isoflavone derivatives to attenuate a-tocopheroxyl radicals in cetyltrimethyl ammonium chloride and sodium dodecyl sulphate micelles.

Methods

The a-tocopheroxyl radical attenuating ability (TRAA) assay has been described in detail by Witting et al. (1997). Briefly, one hundred mM stock solutions of cetyltrimethyl ammonium chloride (HTAC) and sodium dodecyl sulphate (SDS) were prepared in phosphate buffer. Micellular dispersions of a-tocopherol were prepared by diluting an ethanolic solution of a-tocopherol (0.2 M) into micelles at a final vitamin concentration of 500 mM. This resulting solution was sonicated for 15 sec at which time it was completely homogenous.

LDL was isolated by ultracentrifugation of freshly heparinized plasma obtained from non-fasted healthy male donor (27 years of age). LDL was obtained by direct aspiration and stored at 4° C. for 16 hr before use. Immediately prior to use, excess KBr and remaining low molecular weight water-soluble antioxidants were removed from the LDL by gel filtration chromatography, using a PD-10 column (Pharmacia, Uppsala, Sweden) and the concentration of LDL was determined as described by Lowry et al (1951).

Aliquots of the a-tocopherol-containing micells were placed into the neck of an ESR flat cell (100 mL) and placed 0.5 m from a 125 W Osram HQL-Mercury fluorescent bulb used as a UV light source. To increase the light intensity, the frosted casing of the bulb was removed. Samples were irradiated for 3 min, followed by thorough mixing and subsequent transfer of the flat cell to the corresponding temperature-controlled Dewar insert in the ESR cavity, where the sample was allowed to equilibrate to 37° C. This procedure afforded a-tocopheroxyl radical (a-TO) between 1 and 2 mM as estimated against a 10 mM TPDI nitroxide standard. ESR spectra were obtained at 9.41 GHz with modulation amplitude 1.0 G, microwave power 20 nW, and modulation frequency 12.5 kHz using a Bruker ESP 300 ESR spectrometer fitted with an X-band cavity.

After accumulation of the T=0 min spectrum, the flat cell arrangement containing a-TO was removed from the ESR cavity and the solution was gently coaxed into the neck of the flat cell under positive pressure. The compound of choice was then added to give a final concentration of 10 mM and the treated sample was replaced in the cavity and allowed to equilibrate to standard conditions, and sampling was resumed. The time-dependent decay of ESR signal intensity for a-TO was measured both in the presence and absence of the added co-antioxidant (10 mM) using a sweep time of 20.5 sec, averaging the output from three successive sweeps at each time point, and averaging the results of three separate experiments.

Results

Results are expressed as the relative rate constant of decay of a-tocopheroxyl radicals in the presence of the test sample divided by the relative rate constant of decay of a-tocopheroxyl radicals in the absence of the test sample. TRAA approaching unity is considered to have poor synergistic activity, whereas active compounds show large values because they eliminate the a-tocopheroxyl radicals immediately upon mixing. Ascorbate was used as a positive control.

It was found that Cpd. 12 induced high rate of decay and was active at eliminating a-tocopheroxyl radicals. Results are summarized in Table 6.

TABLE 6
Antioxidant activity of the isoflavone derivatives, expressed as
the ability of the compound to eliminate a-tocopheroxyl radicals. Note:
active compounds show large values.

| | | |
|---|---|---|
| | SDS Micelle | HTAC Micelle |
| Conc. | (negative) | (positive) |

Compound

| | | | |
|---|---|---|---|
| Ascorbate | 10 μM | Immediate decay | Immediate decay |
| Promensil | 0.04 mg/ml | 16.5; 22.1 | 7.4; 11.5 |
| Derivatives | | | |
| Cpd. 5 | 10 μM | 0.12; 2.6 | 5.3; 5.4 |
| Cpd. 7 | 10 μM | 1.9 ± 0.2 | 1.4 ± 0.5 |
| Cpd. 8 | 10 μM | 1.26; 1.58 | 2.37 |
| Cpd. 12 | 10 μM | Almost immediate | 26.77; 25.70 |

It is likely however that oxidation reactions in addition to those catalyzed by tocopheroxyl radicals are important. We therefore investigated whether the derivatives possess additional antioxidant activities.

4.5 Peroxyl Radical Scavenging.

The efficacy of the isoflavone derivatives to inhibit linoleate oxidation induced by peroxyl radicals (in the absence of Vitamin E) was be tested. This test provided information on whether the derivatives have radical scavenging activity independent of Vitamin E.

Methods.

Peroxyl radicals are natural side-products of a number of enzymes, including lipoxygenases and cyclo-oxygenase. Peroxyl radicals were generated by the thermo-labile azo-initiator AAPH. AAPH-induced oxidation of linoleate was performed as described previously (Pryor et al., 1993), except that a lower linoleate concentration was used and SDS was omitted. An aliquot of a linoleate stock solution was added to PBS at 37° C. to give a final concentration of 500 mM. Oxidation was initiated by the addition of AAPH (87.5 mM final concentration) in the absence and presence of either 10 mM of Cpd. 7, Cpd. 8 or Cpd. 12 or 500 mM ascorbate (positive control). Formation of linoleate hydroperoxide was monitored by UV 234 nm using a Perkin Elmer's UV/Vis Lambda 40 spectrophotometer. The test was performed twice as single analysis. An appropriate concentration of alcohol (ethanol and isopropanol; 0.13%-0.2% v/v) was included in the respective control samples.

Dubecco's PBS was purchased from Sigma, St. Louis, Mo. The water-soluble azo peroxyl radical generator, 2,2′-azobis(2-amidinopropane)dihydrochloride (AAPH) was purchased from Polysciences, Warrington, Pa. Ascorbate (sodium salt) was purchased from Merck. AAPH (350 mM) stock solutions were prepared by serial dilution with Dubecco's PBS. Stock solutions of the test compounds (10 mM) were prepared as ethanolic solutions while stock solution of linoleate (300 mM) was made by dissolving in isopropanol.

Results.

It was found that the inclusion of 10 mM Cpd. 12 significantly inhibited the time-dependent increase in 234 nm absorbency induced by exposure of linoleic acid to AAPH, indicating that Cpd. 12 is an efficient scavenger of alkyl peroxyl radicals. Comparable results were obtained in two separate experiments. Ascorbate (used as a positive control) prevented AAPH-induced oxidation of linoleic acid, consistent with existing literature.

4.6 Prevention of Peroxidase-Induced Oxidation of Linoleate.

The aim of this study was to test whether the isoflavone derivatives can affect heme-catalyzed oxidation reactions. Such oxidation reactions are thought to be relevant in vivo and can take place using lipid, protein and/or DNA as substrate(s).

Methods.

The test is similar to the peroxyl scavenging described in section 4.5 above, except that horseradish peroxidase (HRP) plus hydrogen peroxide (H₂O₂), were used instead of the peroxyl radical generator. Increased level of heme is considered as a potential risk factor for atherosclerosis, and heme-containing proteins may be involved in the oxidative modification of LDL. In the presence of H₂O₂, heme proteins give rise to a powerful oxidant (referred to as compound I) that can oxidatively modify lipids, including those in LDL. We therefore tested the ability of the isoflavone to inhibit such lipid oxidation using horse radish peroxidase as the model heme protein. HRP/H₂O₂-induced oxidation of linoleate was as described principally by Witting et al (1997) by replacing LDL with linoleate. Linoleate stock was added to PBS at 37° C. to give a final concentration of 300 mM and oxidized by HRP (40 U/mL)/H₂O₂ (2 mM) in the absence and presence of either 10 mM of Cpd. 7, Cpd. 8 or Cpd. 12, or 25 mM BHT (positive control). Formation of linoleate hydroperoxide was monitored by UV 234 nm using the Perkin Elmer's UV/Vis Lambda 40 spectrophotometer. The test was performed twice as single analysis. An appropriate concentration of alcohol (ethanol and isopropanol; 0.13% -0.2% v/v) was included in the respective control samples.

Dubecco's PBS, butylated hydroxytoluene (BHT) and acid free linoleate, were purchased from Sigma, St. Louis, Mo. Horse radish peroxidase (HRP; Grad II; 100,000 U/502.5 mg lyop. powder) was purchased from Boehringer Mannheim, Germany. Hydrogen peroxide (H₂O₂; 30% w/v) was purchased from Merck. HRP (6000 U/mL) and H₂O₂ (1M) stock solutions were prepared by serial dilution with Dubecco's PBS. Stock solutions of BHT (25 mM) and the test compounds (10 mM) were prepared as ethanolic solutions while stock solution of linoleate (300 mM) was made by dissolving in isopropanol.

Results.

A study was performed to test whether isoflavones are able to react with the strong oxidant compound I. Thus, horse radish peroxidase plus H₂O₂ were exposed to the isoflavone derivatives (Cpd. 7, Cpd. 8 or Cpd. 12). It was found that both Cpd. 7 and Cpd. 8 were able to inhibit linoleic acid oxidation by compound I as effectively as 25 mM BHT (used as positive control). Surprisingly, the inhibitory activity of Cpd. 7 and Cpd. 8 was greater than of an equimolar amount of Cpd. 12, suggesting that their mechanism of action might be complex.

4.7 Inhibition of Lipoxygeitase.

Lipoxygenase is a potentially pro-inflammatory enzyme that that requires oxidative activation and releases peroxyl radicals upon catalytic action. The isoflavone derivatives were tested for their ability to affect lipoxygenase activity in vitro, using both soybean and rabbit reticulocyte 15-lipoxygenase and linoleic acid as substrate.

Methods.

15-Lipoxygenase has been suggested to be involved in the initial stages of in vivo LDL oxidation and has been suggested to contribute to atherogenesis (Kuhn et al., 1994; Folcik et al., 1995). Soybean 15-lipoxygenase (SLO) is a suitable model of 15-lipoxygenase and was therefore used to assess the ability of Cpd. 7, Cpd. 8 and Cpd. 12 to inhibit this enzyme. SLO-induced oxidation of linoleate was performed as described previously (Upston et al., 1996). Briefly, linoleate (100 mM) in PBS was oxidized by SLO (60 U/mL) in the absence and presence of 10 mM Cpd. 7, Cpd. 8 or Cpd. 12, or 100 mM eicosatetraynoic acid (ETYA, positive control). Formation of linoleate hydroperoxide was monitored by UV 234 nm using the Perkin Elmer's UV/Vis Lambda 40 spectrophotomneter. The test was performed twice as single analysis. An appropriate concentration of alcohol (ethanol and isopropanol; 0.13% -0.2% v/v) was included in the respective control samples.

Dubecco's PBS and acid free linoleate, soybean 15-lipoxygenase (SLO; 630,000 U/mg protein) were purchased from Sigma, St. Louis, Mo. Ascorbate (sodium salt) and eicosatetraynoic (ETYA) were respectively purchased from Aldrich and Cayman Chemicals. SLO (12,000 U/mL) and ascorbate (100 mM) stock solutions were prepared by serial dilution with Dubecco's PBS. Stock solutions of ETYA (33.3 mM) and the test compounds (10 mM) were prepared as ethanolic solutions while stock solution of linoleate (300 mM) was made by dissolving in isopropanol.

Results.

It was found that Cpd. 12 is an effective inhibitor of lipoxygenase type-1, as indicated by the strongly attenuated increase in 234 nm absorbance. In fact, even at 10 mM, Cpd. 12 showed comparable inhibitory action than 100 mM ETYA, a well-established inhibitor of lipoxygenase. As for peroxyl radicals, neither Cpd. 7 nor Cpd. 8 were able to inhibit SLO.

5.0 Lipid Studies.

5.1 Distribution of Isoflavones in Lipoprotein Subfractions

The effect of dietary soy protein on blood cholesterol concentrations in the rabbit was first reported in the 1940s by Meeker and Kesten (see review by Kristchevsky, 1995). Subsequently, evidence for an independent effect of isoflavones on blood cholesterol concentrations has been demonstrated in various models including rat, hamsters, nonhuman primates and humans (Carroll, 1991; Anderson et al.,1995; Potter, 1996; Balmir et al., 1996; Clarkson et al., 1998). In humans, Cassidy et al (1995) have reported that 45 mg of isoflavones, but not 23 mg isoflavones, resulted in significant reduction in total and LDL cholesterol concentrations in young females. Similar findings were reported by Potter et al (1993) and Bakhit et al (1994). In contrast, Nestel et al (1997) reported no significant effect on blood lipid concentrations of 45 mg of genistein (Cpd. 3) administered over 5 to 10 weeks period.

In order to confirm the relevance isoflavones in the regulation of blood cholesterol, experiments were performed to investigate the affinity of the isoflavone derivatives for LDL and HDL in serum. In the preliminary study, results of which are shown below, only one compound, Cpd. 1 (daidzein), was used.

Methods.

Cpd. 1 (10 mM and 50 mM), was incubated with whole plasma for 0 h, 4 h and 8 h. The different subfractions LDL/VLDL, HDL and protein (lipoprotein free) were then analysed for their Cpd. 1 concentration. Cpd. 1 was used because it had the most similar polarity to the isoflavone synthetic derivatives.

Results.

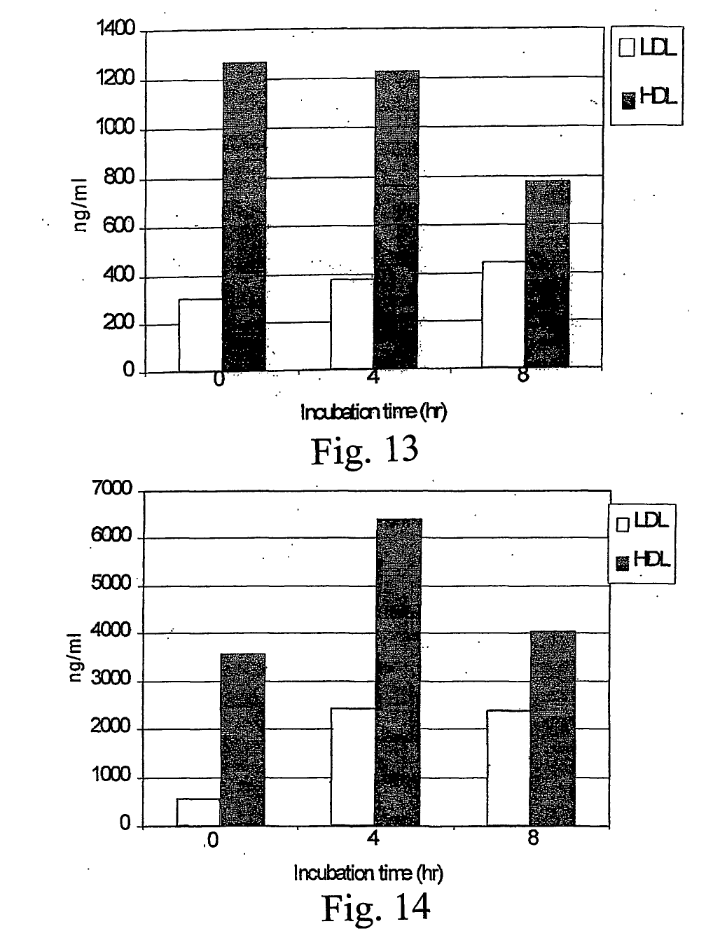

It was found that a small but significant amounts of Cpd. 1 (daidzein) associated with the lipoproteins. 2% of Cpd. 1 associated with the LDL, 6% was found to associate with the HDL and 92% associated with the protein fraction. The amount associated with the lipoproteins increased with time, but not with concentration. The results are demonstrated in FIG. 13 (Cpd. 1, 10 mM) and FIG. 14 (Cpd. 1, 50 mM).

5.2 Regulation of LDL Receptors.

A high risk factor for cardiovascular disease is an elevated LDL cholesterol level. Excess cholesterol results in a greater chance of oxidative damage, and therefore atherosclerosis. Estrogens have a number of potentially beneficial effects on the development of atherosclerosis and the outcome of cardiovascular events. 170-estradiol has been shown to increase LDL receptor activity. There is also evidence that plant-derived estrogens (i.e., phytoestrogens such as isoflavones) may have a protective activity against the incidence of cardiovascular disease, although the underlying mechanism for this remains unknown. Given the structural similarity of isoflavones and their derivatives with certain moieties of estrogen, it is of interest to test whether isoflavones and related compounds may have similar molecular actions. Some studies have suggested that the mechanism by which isoflavones (and estrogens) improve blood lipid profile may be via alterations in LDL receptors quantity or activity (Baum et al., 1998; Kirk et al., 1998). It remains to be established whether the isoflavone derivatives increase the metabolic turnover of cholesterol and increase its removal from the blood stream, by affecting LDL receptor activity. Thus, studies were performed to test the ability of Cpd. 7, Cpd. 8 and Cpd. 12 to stimulate LDL receptor binding on human hepatocyte in vitro by examining the specific binding of labelled LDL in control cells versus cells pre-treated with the test compounds.

Methods.

Confluent human hepatoma (HepG2) cells were preincubated in the absence or presence of the test compound (Cpd. 7, Cpd. 8 and Cpd. 12 at final concentration of 50 nM, 500 nM and 10 mM) for 48 hours, in complete DMEM medium containing 10% fetal bovine serum (FBS). Serum deprivation (i.e. 0.5% FBS) was used as a positive control to induce LDL receptor expression. Following preincubation, cells were washed before 50 mg of [¹²⁵I]-LDL (specific activity 6.5-29.5 cpm/fmol) was added and the cells incubated at 4° C. for another 4 hours in the absence (for total binding) and presence of 500 mg unlabelled LDL (to assess non-specific binding). Cells were then washed, lysed (0.1 M NaOH), and the radioactivity and protein content (BCA assay) of the lysate determined. The number of LDL receptors was determined from the specific binding (total binding minus non-specific binding), assuming one molecule of LDL binds per LDL receptor. Three independent experiments (each in triplicate) were performed with different batches of cells and [¹²⁵I]-LDL.

Results.

To determine the concentration of LDL needed to reach binding saturation, confluent HepG2 cells were incubated in 12-well plates with increasing concentrations of [¹²⁵I]-LDL in the presence of excess (i.e., 500 mg) unlabelled LDL. Saturation was reached at around 50 mg [¹²⁵I]-LDL. Using this concentration, the time required to reach saturation binding at 4° C. was determined to be approximately 4 hours. These established optimal conditions were used for all subsequent binding assays.

Overall, the number of LDL receptors determined in the different sets of experiments varied (Table 7).

TABLE 7
Effect of isoflavone derivatives on LDL receptor expression on
HepG2 cells (mean ± SD, n = 3). All results are expressed
as fmol LDL receptors per mg cell protein

| | | |
|---|---|---|
| Experiment 1 | Experiment 2 | Experiment 3 |

Compound

| | | | |
|---|---|---|---|
| Control | 283 ± 32 | 261 ± 57 | 130 ± 18 |
| 0.5% FBS | 428 ± 41 | 356 ± 32 | 250 ± 22 |
| (+ve control) | | | |
| 50 nM Cpd. 7 | 299 ± 45 | 239 ± 49 | 159 ± 30 |
| 500 nM Cpd. 7 | 281 ± 22 | 199 ± 58 | 171 ± 14 |
| 10 μM Cpd. 7 | 332 ± 46 | 306 ± 70 | 194 ± 46 |
| 50 nM Cpd. 8 | 273 ± 22 | 291 ± 66 | 152 ± 24 |
| 500 nM Cpd. 8 | 312 ± 42 | 291 ± 52 | 193 ± 18 |
| 10 μM Cpd. 8 | 315 ± 24 | 273 ± 57 | 143 ± 34 |
| 50 nM Cpd. 12 | 319 ± 43 | 134 ± 52 | 156 ± 24 |
| 500 nM Cpd. 12 | 311 ± 35 | 233 ± 40 | 136 ± 23 |
| 10 μM Cpd. 12 | 290 ± 42 | 161 ± 35 | 174 ± 35 |

As can be seen from the results expressed as an incremental increase in LDL receptor expression over control, Cpd. 12 was without consistent effect, while Cpd. 7 and Cpd. 8 increased LDL binding. In the case of Cpd. 7 (but not Cpd. 8) the expression of LDL receptor increased concentration-dependently although the extent of this increase was small when compared to that obtained with serum deprivation. Cpd. 8 had no clear concentration-dependent effect.

Although the effects observed were small, at least compared with serum deprivation, the increase in LDL binding was observed consistently in separate sets of experiments using different batches of both cells and LDL, suggesting that the observations made reflects a true activity of Cpd. 7 and Cpd. 8. These results show the potential for isoflavones and derivatives thereof to prevent or impede the accelerated development and progression of atherosclerotic plagues and associated with restenosis.

### EXAMPLE 4

### Angiogenesis Activity

Angiogenesis occurs through a number of distinct steps that can be defined as endothelial cell activation, migration, proliferation, and reorganisation to form the mature vessel characterised by a lumen. The effect of isoflavone derivatives on angiogenesis and in particular endothelial cell proliferation and endothelial cell migration uses assessed by in vitro assays.

4.1 Effect of Isoflavone Derivatives on Endothelial Cell Proliferation.

Methods:

Cell Preparation

Human umbilical vein endothelial cells between passage 1 and 3 were used. Cells were grown in gelatin coated flasks in medium 199 with Earle's Salts supplemented with 20% foetal calf serum, 25 mg/ml endothelial cell growth supplement and 25 mg/ml heparin. Cells from two different donors were used.

Proliferation Assay

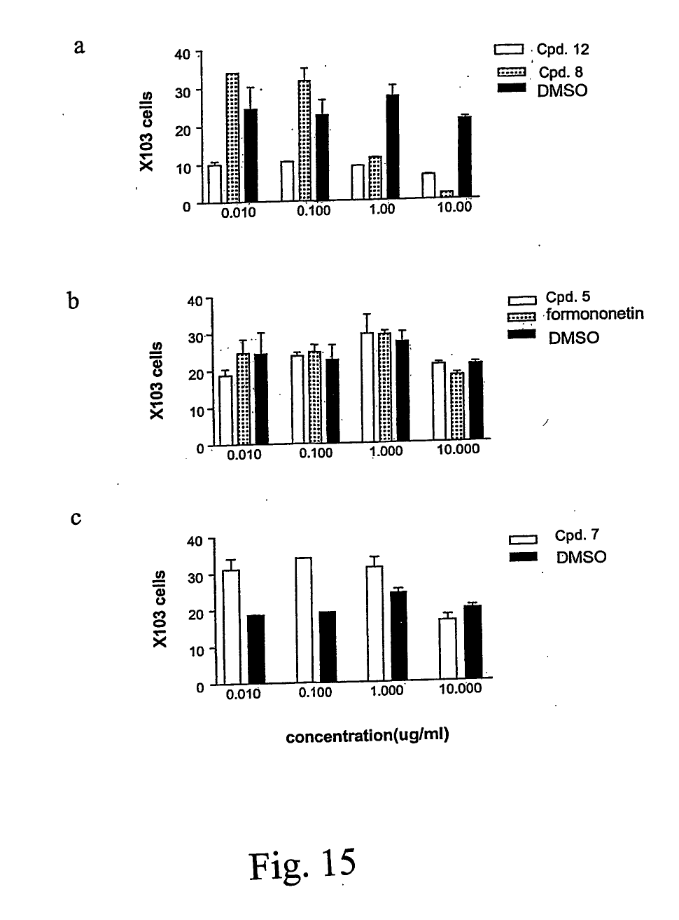

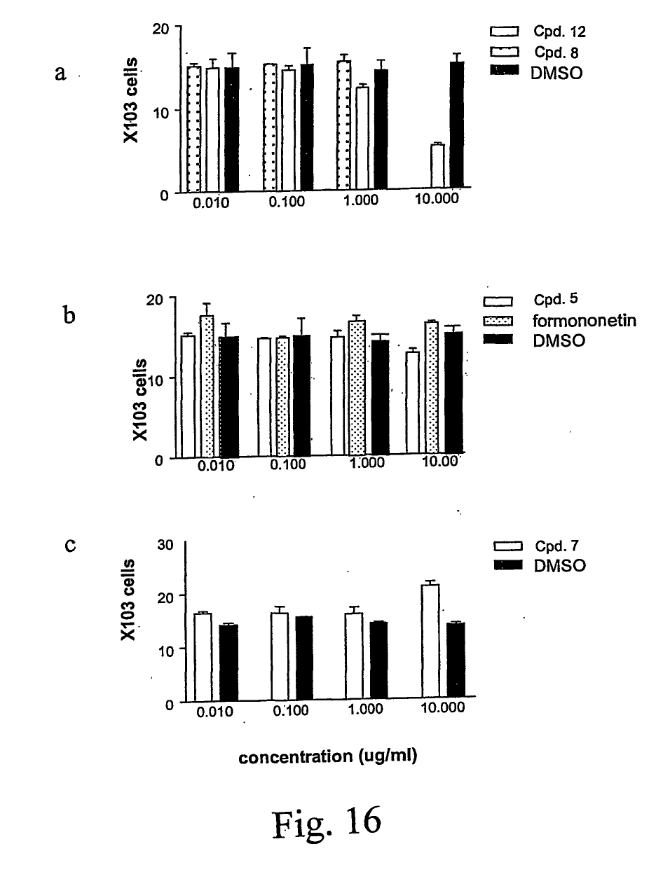

5×103 cells in 100 ml growth medium were plated into gelatin coated microtitre wells. The test compounds were added at plating. The cells were then incubated for 3 days and cell number determined by a calorimetric assay (CellTiter96 Aqueous assay, Promega). Results were compared to a standard curve performed in each experiment. Two different endothelial cell isolates were used and the assays were performed on two different days. The effect of isoflavone derivatives on the proliferation of human umbilical vein endothelial cells are shown for donor No. 1 (FIG. 15) and donor No. 2 (FIG. 16).

Results:

FIGS. 15 and 16 show the effect of Cpd. 5, Cpd. 7, Cpd. 8 and Cpd. 12 on endothelial cell proliferation. Both Cpd. 8 (1-10 mg/ml) and Cpd. 12 (0.01-10 mg/ml) inhibited cellular proliferation and did not cause toxicity. Interestingly, there was considerable line variation in the responsiveness of the cells to these compounds (FIG. 15a and FIG. 16a). Cpd. 5 (0.01-10 mg/ml) was found to have no apparent effect on endothelial cell proliferation, in either of the two cell isolates studied (FIG. 15b and FIG. 16b). Similarly, Cpd. 7 (0.01-10 mg/ml) displayed no appreciable antiproliferative action on the two cell isolates studied (FIG. 15c and FIG. 16c).

4.2 Effect of Isoflavone Derivatives on Migration of Endothelial Cells.

Methods:

Cell Preparation

Human umbilical vein endothelial cells between passage 1 and 3 were used. They were grown in gelatin coated flasks in medium 199 with Earle's Salts supplemented with 20% foetal calf serum, 25 mg/ml endothelial cell growth supplement and 25 mg/ml heparin.

Migration Assay

5×105 cells per well in growth medium were plated onto fibronectin coated 6 well trays and grown to confluence. A wound was produced along the monolayer with the use of a cell scraper (Costar). The cells were then washed once and fresh medium with the test compounds was added. Defined points along the wound were marked using an ink condenser (Olympus microscopes) allowing the movement of cells to be visualized at constant points over the next 48 hours. Photographs were taken at relevant times and the degree of cell movement from the wound front assessed.

Results:

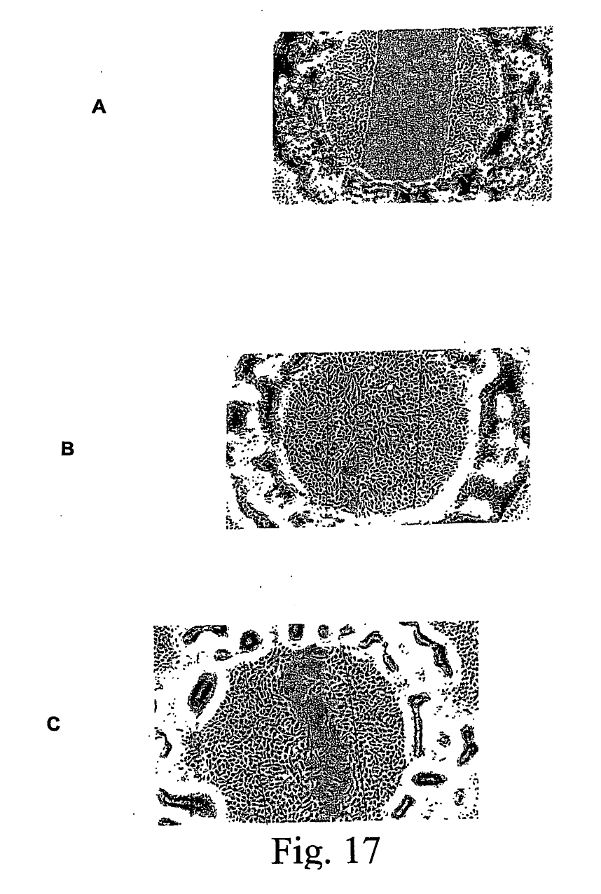

FIG. 17 shows the effect of Cpd. 12 on endothelial cells migration. The photos show the wound at baseline (time 0 hr) (FIG. 17a) and the amount of cell movement 30 hours after wounding. Treatment with Cpd. 12 (1 mg/ml final concentration), markedly inhibited endothelial cell migration towards the wound after 30 hours (Figure c). DMSO (solvent control) had no effect on proliferation and after 30 hours of treatment cells migrated towards the wound to formed a monolayer (FIG. 17b). Photos were taken at different points along the wound but the points were constant over the course of the experiment. The initial wound front is marked.

The results of this example show that Cpd. 8 and Cpd. 12 inhibited endothelial cell proliferation whilst Cpd. 12 inhibited endothelial cell migration. Thus isoflavones and derivatives thereof show potential for use as cardioprotective therapeutics, and in particular after vascular intervention and surgery.

### EXAMPLE 5

### Inhibition of Cell Adhesion Molecule Expression

The ability of isoflavone compounds and derivatives to inhibit the expression of E-selectin was measured by the protocol set out in Litwin et al. (1997) and is incorporated herein by reference.

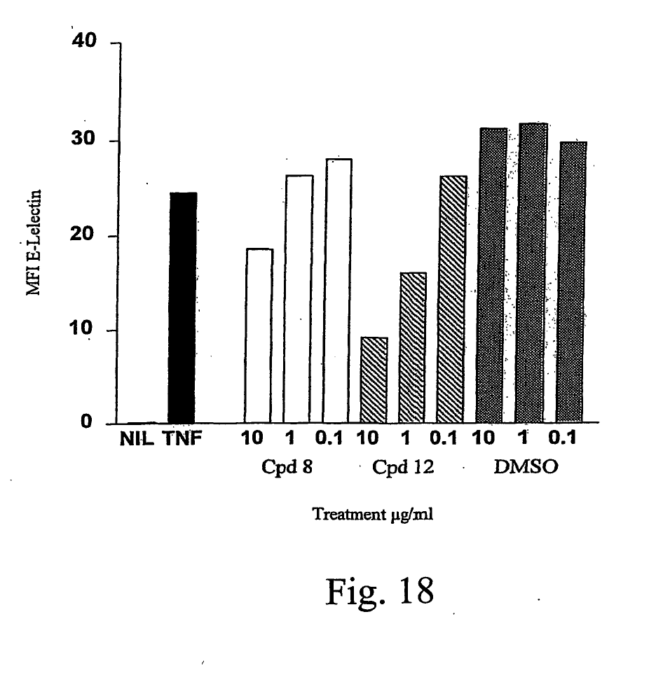

FIG. 18 provides comparative data on the ability of a number of isoflavone compounds to inhibit the expression of E-selectin conducted in accordance with the method of Litwin et al. The interaction of isoflavone metabolite Cpds. 8 and 12 on human umbilical vein endothelial cells, activated by INF-α, was measured against the concentration of the compound. The cells were pre-incubated for either 2 or 18 hours prior to TNF stimulation. In comparison with a DMSO control, isoflavone Cpd. 8 showed very good inhibition of E-selectin expression, especially at 10 μg/ml. Isoflavone Cpd. 12 showed excellent inhibition of E-selectin expression, especially at concentrations of 1 and 10 μg/ml.

The example establishes that the claimed isoflavones and derivatives specifically block the ability of E-selectin to be expressed by vascular endothelial cells in response to many signals known to be active in restenosis, inflammatory response and other diseases mediated by cell adhesion molecule expression.

### EXAMPLE 6

### Restenosis Model—Cellular migration and proliferation

The key feature of atherosclerosis in humans is vascular smooth muscle cell (VSMC) migration from the arterial media to the intima, followed by proliferation within the intima. The animal model of atherosclerosis used in this experiment aimed to induce VSMC migration and neointimal proliferation by mechanical damage to the endothelium. A probe was introduced into the femoral artery of the mouse and was passed through the iliac artery to the bifurcation of the aorta. The probe was then withdrawn and the femoral artery ligated to prevent haemorrhage. Colateral circulation prevents any limb-associated ischaemia. Neointimal proliferation occurred in the iliac artery over the following 4 weeks.

Proliferation was enhanced by feeding the mouse cholesterol during this time (commencing one week before surgery). At four weeks, the mice were sacrificed and the iliac vessels on both sides are harvested (the non-operated vessel was used as a control). Neointimal proliferation is expressed as the ratio of the intimal area to the intimal plus media area.

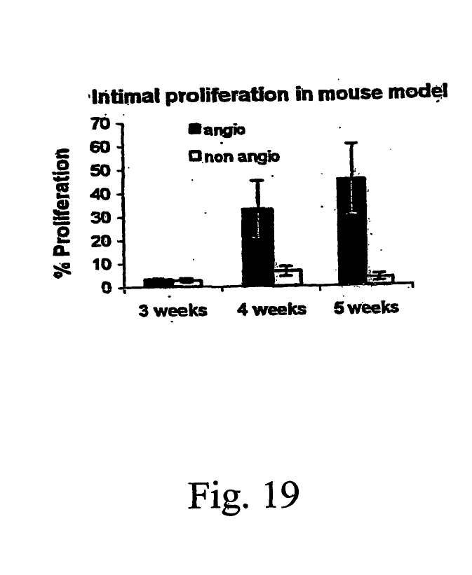

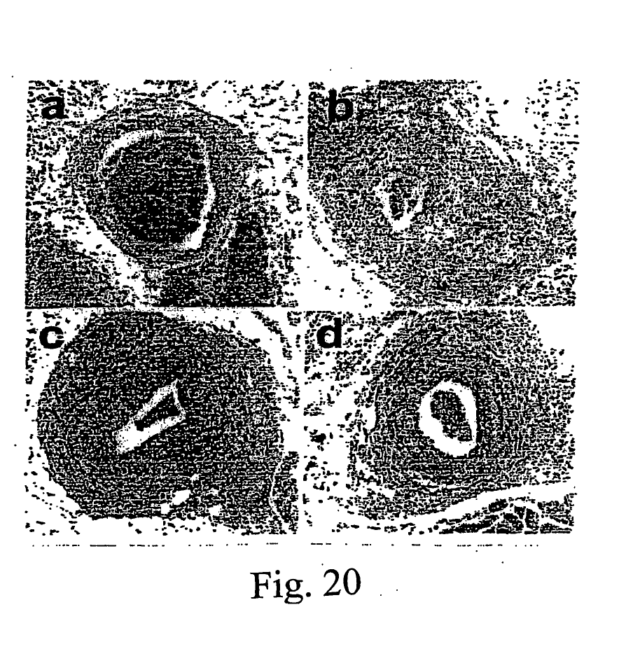

This technique (mainly using gene knockout mice) has been found to be very reproducible using sample sizes of 5-10 mice. FIG. 19 shows a typical profile of proliferation in normal mice. Minimal proliferation is seen at 3 weeks, with a substantial rise in proliferation at 4 weeks, followed by a further rise in week 5. Minimal neointimal proliferation occurs in the control non-operated vessel (“non-angio”) from the opposite side.

The ability of isoflavone derivatives to reduce and/or prevent the “atherosclerotic” response in a mouse model of neointimal hyperplasia was investigated. The compounds tested were Cpd. 5 and Cpd. 12.

Methods

Atherosclerosis was induced in the femoral artery of each mouse by mechanically removing the endothelium using a probe. The ability of isoflavone compounds to inhibit the atherosclerotic response was assessed by comparing treatment and placebo groups. C57/B16 mice (8-10 week old male) were anaesthetized, and the right femoral artery was de-endothilialized using a dissection microscope. These mice were also fed with a 2% cholesterol diet to enhance the progression of the disease. The mice were sacrificed at week 4. Their treated (right) and control (left) arteries were both collected and fixed in formalin for histopathological evaluation.

The mice were fed a diet of 2% cholesterol mixed with normal mouse chow for a week prior to surgery. The treatment groups were fed the chow with Cpd. 5 or Cpd. 12 or Cpds. 5+12. Mice were anaesthetised with Avertin, and angioplasty of the femoral artery was performed. After surgery, the mice were moved onto a warm mat in a quiet, darkened area and their recovery closely monitored. They were housed in sterile cages with sterile bedding to avoid infection. The mice continued a diet of cholesterol mixed with normal mouse chow for the next 4 weeks. The mice were euthanased at 4 weeks postsurgery. Their femoral arteries were collected for pathological examination. Sections were evaluated by image analysis to determine the area of the various sections of the arterial wall, and the numbers of cells in each area. The primary parameter being evaluated was the increase in the intimal area, expressed as the ratio of the area of the intima versus the area of the intima plus media. The interval sections (every 50 μm, or every ten sections) were mounted and stained with H & E. The cross-sectional areas of the endothelium, intima and media were evaluated, using image analysis.

Results

FIG. 20 shows a transverse section through the iliac artery 4 weeks after surgery. FIG. 20a shows the absence of neointimal proliferation in the iliac artery from the non-operated side, where the intima is about one cell thick. FIG. 20b shows substantial neointimal proliferation in the iliac artery in response to surgery, showing a thickness of approximately 50% of the vessel wall. FIGS. 20c and 20d are post-surgical iliac arteries from mice treated with Cpd. 12 and Cpd. 5, respectively. Neointimal proliferation can be seen in each case, but the proliferation was significantly reduced in Cpd. 5.

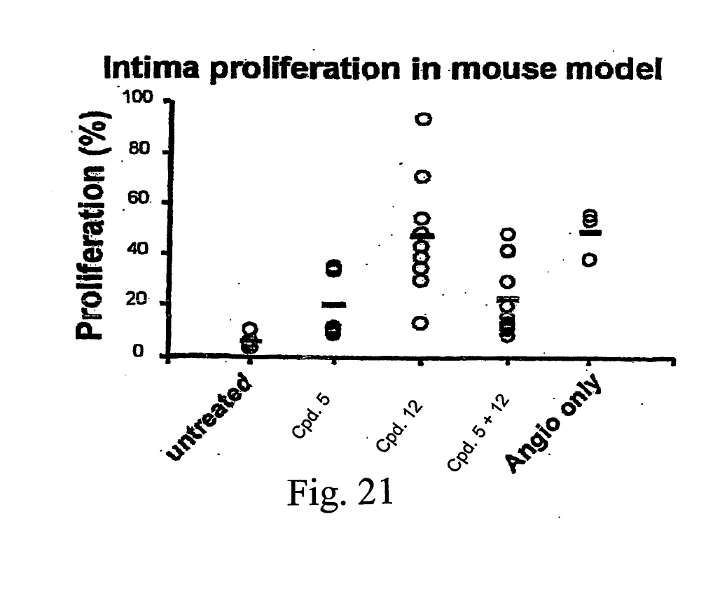

FIG. 21 quantitates the effects of the test compounds on neointimal proliferation, both individually and together. The untreated vessels (no surgery) show 5±1% intimal thickness, while the surgical-treated only vessels show 50±5% intimal thickness. Cpd. 5, both alone and in combination with Cpd. 12 reduced neointimal proliferation to 30±6% and 32±10%, respectively, while Cpd. 12 had no detectable effect of the extent of neointimal proliferation (50±29%). Notably these data are consistent with Cpd. 5 significantly reducing neointimal proliferation, but Cpd. 12 having no appreciable effect.

Notably, the area of the neointimal proliferation corresponds well with the numbers of cells within the intima determined using image analysis.

These results show that isoflavones and derivatives thereof, in particular Cpd. 5, significantly inhibits proliferation of VSMC in the neointimal of the mouse model of atherosclerosis. Neointimal area was reduced by approximately 50% at the time point tested (4 weeks). Cpd. 12 had no appreciable effect on neointimal proliferation in the mouse model at this time point. A combination of Cpd. 5 and Cpd. 12 did not appear to alter the inhibitory effect of seen with Cpd. 5 alone. These results show the potential for isoflavones and derivatives thereof to modulate the migration and progression of VSMC and to prevent or impede the development and progression of atherosclerotic plaques and hence restenosis after mechanical damage, thus providing a benefit in vascular protection.

The invention has been described herein, with reference to certain preferred embodiments, in order to enable the reader to practice the invention without undue experimentation. However, a person having ordinary skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited herein, if any, are hereby incorporated by reference.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification individually or collectively, and any and all combinations of any two or more of said steps or features.

The reference to any prior art in this specification is not, and should not be taken as, an acknowledgment or any form of suggestion that that prior art forms part of the common general knowledge in the field of endeavour.

Bibliography

1. Steinberg D, Parthasarathy S, Carew T E, Khoo J C, Witztum J L

“Beyond cholesterol. Modifications of low-density lipoprotein that increase its atherogenicity”.

N Engl J Med Apr. 6, 1989;320(14):915-24
2. Hillis G S and Flapan A D

“Cell adhesion molecules in cardiovascular disease: a clinical perspective”.

Heart 1998 May;79(5):429-31
3. Greene, T. W.,

“Protective Groups in Organic Synthesis”

John Wiley & Sons, New York (1981)
4. Goodman and Gilman

“The pharmacological basis of therapeuties, 1299”

7th Edition, 1985
5. Brown, L., and Langer, R.,

“Transdermal delivery of drugs”

Annual Review of Medicine, 39:221-229 (1989)
6. Panchagnula R, Pillai O, Nair V B, Ramarao P.

“Transdermal iontophoresis revisited”

Curr. Opin. Chem. Biol. 2000 August; 4(4):468-73
7. Chang et al. (1994)

Microwave-Mediated Synthesis of Anticarcinogenic Isoflavones from Soybeans

J Agric Food Chem 1994, 42. 1869-1871
8. WO 98/08503

Novogen Research Pty Limited

“Therapeutic methods and compositions involving isoflavones”
9. WO 00/49009

Novogen Research Pty Limited

“Production of isoflavone derivatives”
10. Pryor D B, Shaw L, McCants C B, Lee K I, Mark D B, Harrell F E, Muhlbaier L H, Califf R M

“Value of the history and physical in identifying patients at increased risk for coronary artery disease”.

Ann Intern Med Jan. 15, 1993;118(2):81-90
11. Witting P K, Upston J M, Stocker R

“Role of alpha-tocopherol radical in the initiation of lipid peroxidation in human lipoprotein exposed to horse radish peroxidase”.

Biochemistry Feb. 11, 1997;36(6):1251-8
12. Kuhn H, Belkner J, Zaiss S, Fahrenklemper T, Wohlfeil S

“Involvement of 15-lipoxygenase in early stages of atherogenesis”

J Exp Med Jun. 1;179(6):1903-11
13. Folcik V A, Nivar-Aristy R A, Krajewski L P, Cathcart M K

“Lipoxygenase contributes to the oxidation of lipids in human atherosclerotic plaques”.

J Clin Invest 1995 July;96(1):504-10
14. Upston J M, Neuzil J, Stocker R

“Oxidation of LDL by recombinant human 15-lipoxygenase: evidence for alpha-tocopherol-dependent oxidation of esterifed core and surface lipids”.

J Lipid Res 1996 December;37(12):2650-61
15. Kritchevsky D

“Dietary protein, cholesterol and atherosclerosis: a review of the early history”

J Nutr 1995 March;125 (3 Suppl):589S-593S
16. Carroll K K

“Review of clinical studies on cholesterol-lowering response to soy protein”.

J Am Diet Assoc 1991 July;91(7):820-7
17. Anderson J W, Johnstone B M, Cook-Newell M E

“Meta-analysis of the effects of soy protein intake on serum lipids”

New Engl J Med Aug. 3, 1995;333(5): 276-82
18. Potter S M

“Soy protein and serum lipids”

Curr Opin Lipidol 1996 August; 7(4):260-4
19. Balmir F, Staack R, Jeffrey E, Jimenez M D, Wang L, Potter S M

“An extract of soy flour influences serum cholesterol and thyroid hormones in rats and hamsters”.

J Nutr 1996 December; 126(12): 3046-53
20. Clarkson T B, Anthony M S

“Phytoestrogens and coronary heart disease”

Bailliere's Clinical Endocrinology and Metabolism 1998 December; 12(4): 589-604
21. Cassidy A, Bingham S, Setchell K

“Biological effects of isoflavones in young women: importance of the chemical compositions of soybean products”

Br J Nutr 1995; 74:587-601
22. Potter S M, Bakhit R M, Essex-Sorlie D, Weingartner K E, Chapman K M, Nelson R A, Prabhudesai M, Savage W D, Nelson A I, Winter L W et al

“Depression of plasma cholesterol in men by consumption of baked products containing soy protein”.

Am J Clin Nutr 1993 October; 58(4): 501-6
23. Bakhit R M, Klein B P, Essex-Sorlie D, Ham J O, Erdman J W, Potter S M

“Intake of 25 g of soybean protein with or without soybean fiver alters plasma lipids in men with elevated cholesterol concentrations”.

J Nutr 1994 February; 124(2): 213-22
24. Nestel P J, Yamashita T, Sasahara T, Pomeroy S, Dart A, Komesaroff P, Owen A, Abbey M

“Soy isoflavones improve systemic arterial compliance but not plasma lipids in menopausal and perimenopausal women”.

Arteriosclerosis, Thrombosis and Vascular Biology 1997December; 17(12): 3392-3398
25. Baum J A, Teng H, Erdman Jr J W, Weigel R M, Klein B P, Persky V W, Freels S, Surya P, Bakhit R M, Ramos E, Slay N F, Potter S M

“Long-term of soy protein improves blood lipid profiles and increases mononuclear cell low-density-lipoprotein receptor messenger RNA in hypercholesterolemic, postmenopausal women”.

Am J Clin Nutr 1998; 68:545-51
26. Kirk E A, Sutherland P, Wang S A, Chait A, LeBoeuf R C

“Dietary isoflavones reduce plasma cholesterol and atherosclerosis in C57BL/6 Mice but LDL receptor-deficient mice”.

J Nutr 1998; 128: 954-959
27. Litwin M, Clark K, Noack L, Berndt M, Albelda S, Vadas M, Gamble J

“Novel cytokine-independent induction of endothelial adhesion molecules regulated by platelet/endothelial cell adhesion molecule (CD31)”

J Cell Biology 6 Oct. 1997; 139(1): 219-228

1. A method for inhibiting expression or activity of an adhesion molecule associated with an endothelial cell, which method comprises the step of contacting the adhesion molecule or endothelial cell with one or more compounds of formula I in an amount sufficient to inhibit said expression or activity, wherein formula I is represented by: 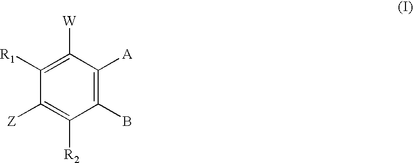
in which
R₁, R₂ and Z are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₂ is as previously defined, and R₁ and Z taken together with the carbon atoms to which they are attached form a five-membered ring selected from 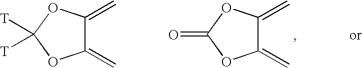
R₁ is as previously defined, and R₂ and Z taken together with the carbon atoms to which they are attached form a five-membered ring selected from 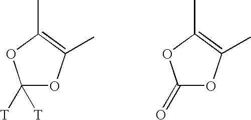
and
W is R₁, A is hydrogen, hydroxy, NR₃R₄ or thio, and B is selected from 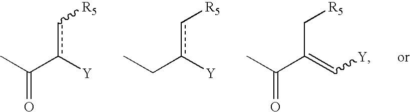
W is R₁, and A and B taken together with the carbon atoms to which they are attached form a six-membered ring selected from 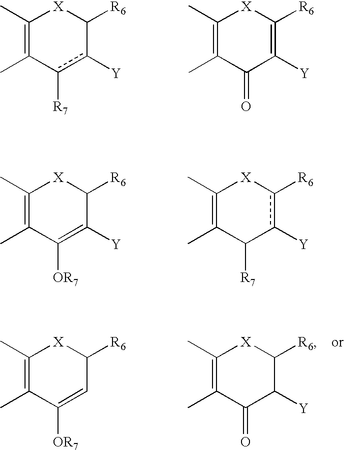
W, A and B taken together with the groups to which they are associated are selected from 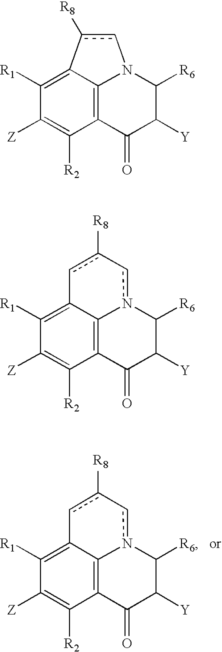
W and A taken together with the groups to which they are associated are selected from 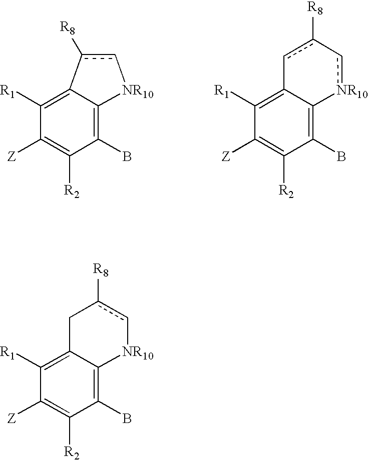
and B is selected from 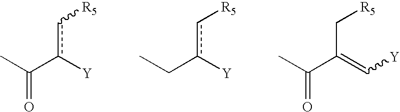
wherein
R₃ is hydrogen, alkyl, arylalkyl, alkenyl, aryl, an amino acid, C(O)R₁₁ where R₁₁ is hydrogen, alkyl, aryl, arylalkyl or an amino acid, or CO₂R₁₂ where R₁₂ is hydrogen, alkyl, haloalkyl, aryl or arylalkyl,
R₄ is hydrogen, alkyl or aryl, or
R₃ and R₄ taken together with the nitrogen to which they are attached comprise pyrrolidinyl or piperidinyl,
R₅ is hydrogen, C(O)R₁₁ where R₁₁ is as previously defined, or CO₂R₁₂ where R₁₂ is as previously defined,
R₆ is hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, alkenyl, aryl, arylalkyl or Si(R₁₃)₃ where each R₁₃ is independently hydrogen, alkyl or aryl,
R₈ is hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino,
the drawing 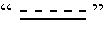
represents either a single bond or a double bond,
T is independently hydrogen, alkyl or aryl,
X is O, NR₄ or S, and
Y is 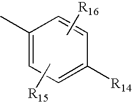
wherein
R₁₄, R₁₅ and R₁₆ are independently hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, thio, alkylthio, amino, alkylamino, dialkylamino, nitro or halo,
including pharmaceutically acceptable salts thereof.

2. A method of claim 1, wherein the compounds of formula I are represented by formulae II-VIII: 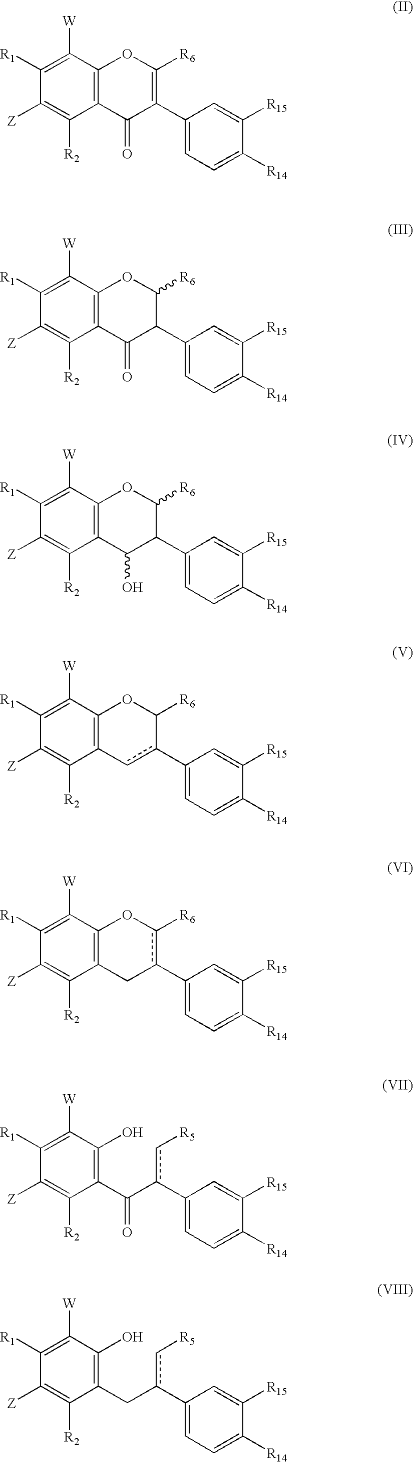
in which
R₁, R₂, R₅, R₆, R₁₄, R₁₅, W and Z are as defined above, in claim 1,
including pharmaceutically acceptable salts thereof.

3. A method of claim 2, wherein
R₁, R₂, R₁₄, R₁₅, W and Z are independently hydrogen, hydroxy, OR₉, OC(O)R₁₀, C(O)R₁₀, COOH, CO₂R₁₀, alky, haloalkyl, arylalkyl, aryl, thio, alkylthio, amino, alkylamino, dialkylamino, nitro or halo,
R₅ is hydrogen, C(O)R₁₁ where R₁₁ is hydrogen, alky, aryl, or an amino acid, or CO₂R₁₂ where R₁₂ is hydrogen, alkyl or aryl,
R₆ is hydrogen, hydroxy, alkyl, aryl, COR₁₁ where R₁₁ is as previously defined, or CO₂R₁₂ where R₁₂ is as previously defined,
R₉ is alkyl, haloalkyl, arylalkyl, or C(O)R₁₁ where R₁₁ is as previously defined, and
R₁₀ is hydrogen, alkyl, amino, aryl, an amino acid, alkylamino or dialkylamino, including pharmaceutically acceptable salts thereof.

4. A method of claim 2, wherein
R₁ and R₁₄ are independently hydroxy, OR₉, OC(O)R₁₀ or halo,
R₂, R₁₅, W and Z are independently hydrogen, hydroxy, OR₉, OC(O)R₁₀, C(O)R₁₀, COOH, CO₂R₁₀, alkyl, haloalkyl, or halo,
R₅ is hydrogen, C(O)R₁₁ where R₁₁ is hydrogen or alkyl, or CO₂R₁₂ where R₁₂ is hydrogen or alkyl,
R₆ is hydrogen or hydroxy,
R₉ is alkyl, arylalkyl or C(O)R₁₁ where R₁₁ is as previously defined, and
R₁₀ is hydrogen or alkyl,
including pharmaceutically acceptable salts thereof.

5. A method of claim 2, wherein
R₁ and R₁₄ are independently hydroxy, methoxy, benzyloxy, acetyloxy or chloro,
R₂, R₁₅, W and Z are independently hydrogen, hydroxy, methoxy, benzyloxy, acetyloxy, methyl, trifuoromethyl or chloro,
R₅ is hydrogen or CO₂R₁₂ where R₁₂ is hydrogen or methyl, and R₆ is hydrogen,
including pharmaceutically acceptable salts thereof.

6. A method of claim 1, wherein the compounds of formula I are selected from: 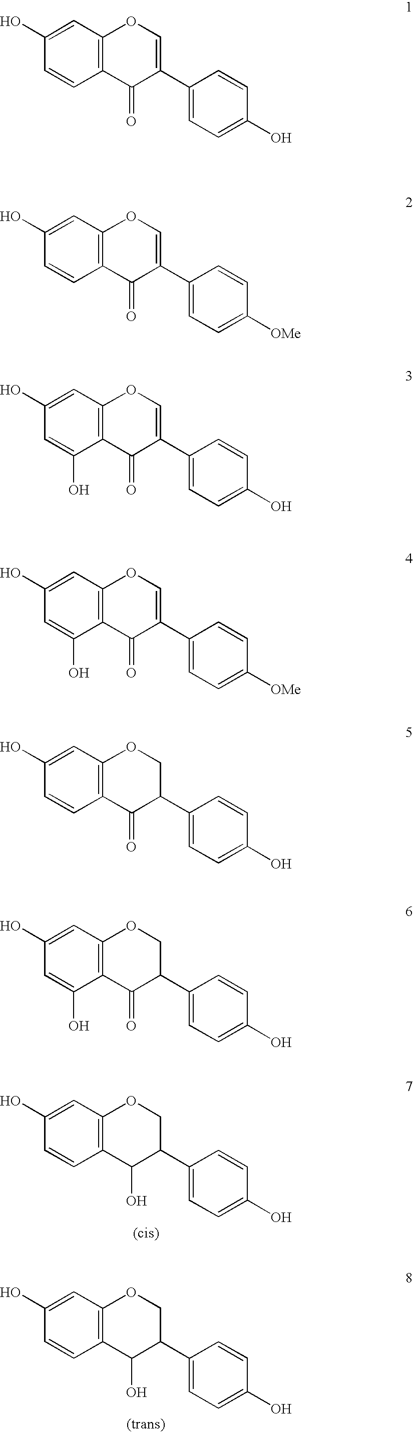 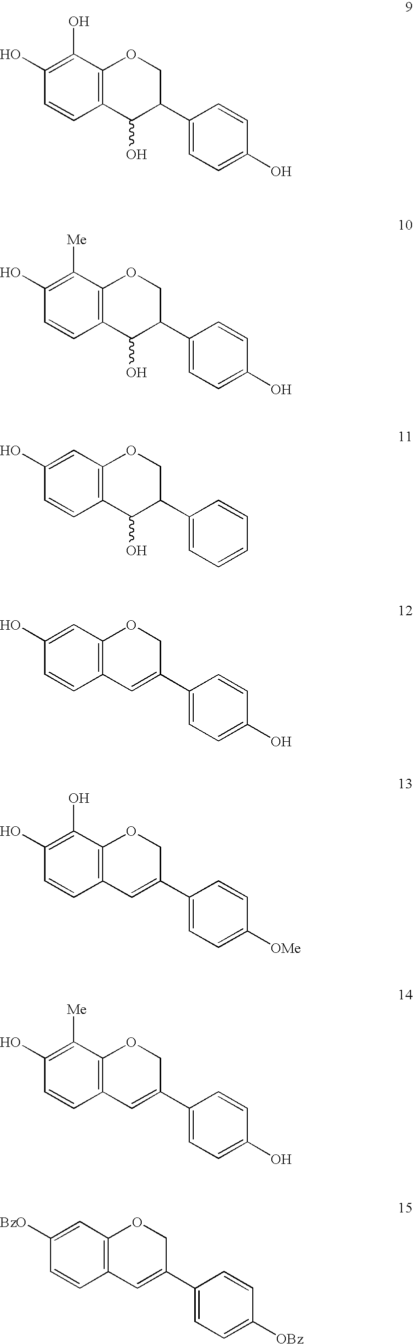 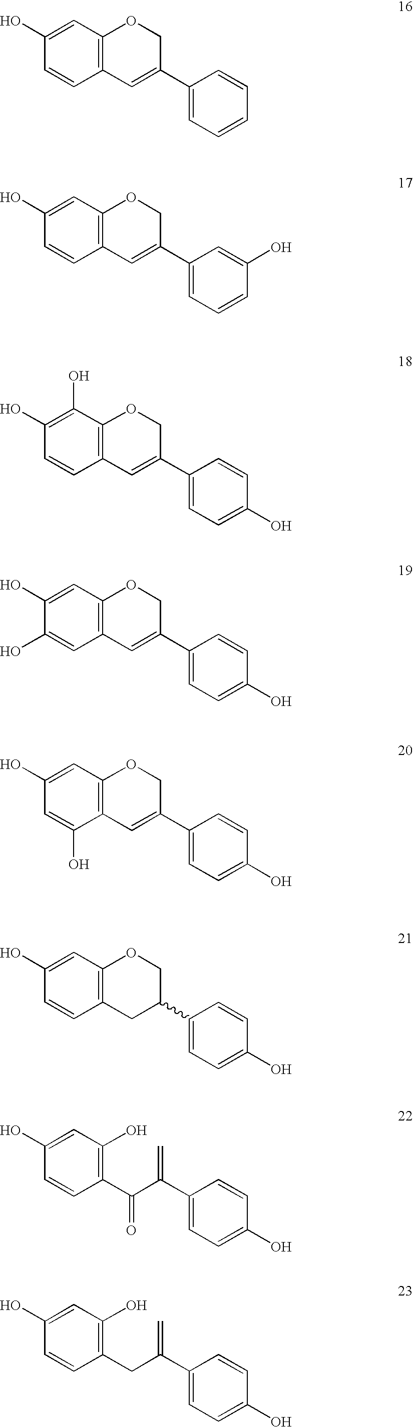 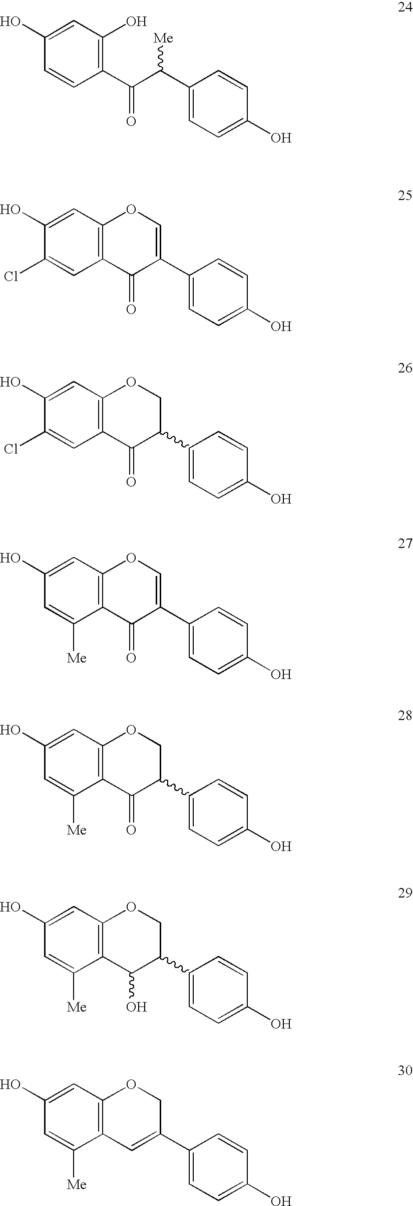
including pharmaceutically acceptable salts thereof.

7. A method of any of claims 1 to 6, wherein the adhesion molecule is E-selectin or vascular cell surface adhesion molecule (VCAM-1).

8. A method for inhibiting the expression or activity of adhesion molecules associated with endothelial cells in a subject, which method comprises the step of administering to the subject a therapeutically effective amount of one or more compounds of formula I as defined in any of claims 1 to 6.

9. A method of claim 8, wherein the adhesion molecule is E-selectin or vascular cell surface adhesion molecule (VCAM-1).

10. A method of claim 8 or claim 9, wherein the subject is a human.

11. A method of treating a disease mediated by expression or activity of adhesion molecules associated with endothelial cells in a subject, which method comprises the step of administering to, the subject one or more compounds of formula I as defined in any of claims 1 to 6 in an amount sufficient to inhibit said expression of activity of the adhesion molecules associated with the endothelial cells.

12. A method of claim 11, wherein the disease is a vascular disease.

13. A method of claim 12, wherein the vascular disease is selected from restenosis, inflammatory disease, coronary artery disease, angina and small vessel disease.

14. A method of claim 13, wherein the vascular disease is post-angioplasty restenosis.

15. A method for the treatment, amelioration, prophylaxis or reduction in the risk of restenosis in a subject, which method comprises the step of administering to the subject a therapeutically effective amount of one or more compounds of formula I as defined in any of claims 1 to 6.

16. A method of claim 15, wherein the restenosis is associated with vascular intervention selected from percutaneous transluminal coronary angioplasty, direction coronary atherectomy and stent.

17. A method for the treatment of procedural vascular trauma in a subject, which method comprises the step of administering to the subject a therapeutically effective amount of one or more compounds of formula I as defined in any of claims 1 to 6.

18. A method of claim 17, wherein the procedural vascular trauma is selected from angioplasty, vascular surgery, graft and transplant procedure.

19. A method for the treatment or prophylaxis of vascular disease in a subject, which method comprises the step of administering to the subject a therapeutically effective amount of one or more compounds of formula I as defined in any of claims 1 to 6.

20. A method of claim 19, wherein the vascular disease is selected from atherosclerosis, restenosis, hypertension, inflammatory disease, coronary artery disease, angina and small vessel disease.

21. A method of claim 20, wherein the vascular disease is post-angioplasty restenosis.

22. A pharmaceutical composition in a dosage form suitable for use in the treatment of a disease mediated by expression or activity of adhesion molecules associated with endothelial cells in a subject, which composition comprises one or more compounds of formula I as defined in any of claims 1 to 6 in association with a pharmaceutical acceptable carrier.

23. A pharmaceutical composition in a dosage form suitable for use in preventing or reducing the risk of vascular disease in a subject, which composition comprises one or more compounds of formula I as defined in any of claims 1 to 6 in association with a pharmaceutical acceptable carrier.

24. Use of one or more compounds of formula I as defined in any of claims 1 to 6 in the manufacture of a medicament for inhibiting the expression or activity of adhesion molecules associated with endothelial cells in a subject.

25. Use of one or more compounds of formula I as defined in any of claims 1 to 6 in the manufacture of a medicament for the treatment of a disease mediated by expression or activity of adhesion molecules associated with endothelial cells.

26. Use of one or more compounds of formula I as defined in any of claims 1 to 6 in the manufacture of a medicament for the treatment, amelioration, prophylaxis or reduction in the risk of restenosis.

27. Use of one or more compounds of formula I as defined in any of claims 1 to 6 in the manufacture of a medicament for the treatment of vascular disease and/or procedural vascular trauma.
